(19) United States
(12) Patent Application Publication
(10) Pub. No.: US 2015/0045560 A1
(43) Pub. Date: Feb. 12, 2015

(54) NOVEL FUSED NAPHTHALENE CYCLOHETERO RING COMPOUNDS, AND METHODS AND USES THEREOF

(71) Applicant: Corning Incorporated, Corning, NY (US)

(72) Inventors: Mingqian He, Horseheads, NY (US); Jieyu Hu, Littleton, CO (US); Weijun Niu, Painted Post, NY (US); Adama Tandia, Nelson, PA (US)

(21) Appl. No.: 14/387,102

(22) PCT Filed: Mar. 28, 2013
(86) PCT No.: PCT/US13/34347

§ 371 Date: Sep. 22, 2014

Related U.S. Application Data
(60) Provisional application No. 61/617,202, filed on Mar. 29, 2012.

(51) Int. Cl. C07F 7/08; H01L 51/00; C07C 211/57; C07D 403/04; C07C 37/16; C07C 303/30

(52) U.S. Cl. CPC C07F 7/083; C07C 37/16; C07C 303/30; C07C 211/57; C07D 403/04; H01L 51/0094; H01L 51/0052; H01L 51/0072
USPC 548/421; 556/489; 556/413; 568/717; 568/33; 564/428

(57) ABSTRACT

Described herein are heterocyclic organic compounds of following formulae: More specifically, described herein are fused heterocyclic naphthalene compounds, polymers based on fused heterocyclic naphthalene compounds, methods for making these compounds, and uses thereof. The compounds described have improved polymerization and stability properties that allow for improved material processibility for use as organic semiconductors (OSCs).

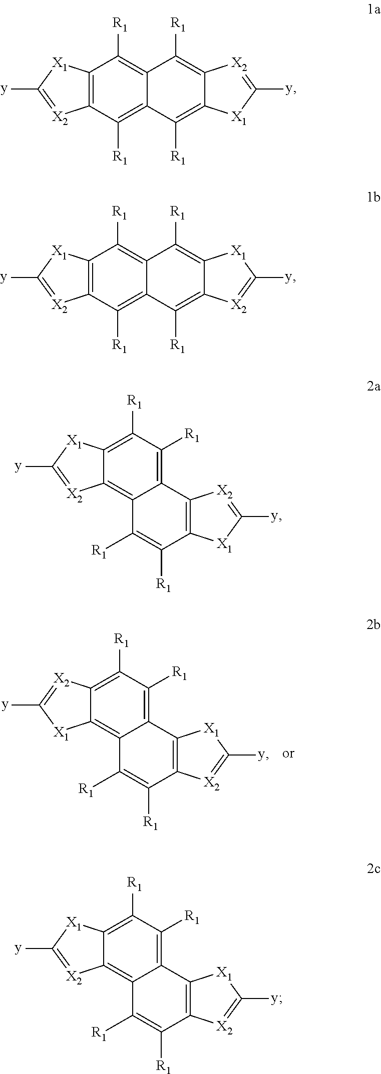

### CROSS-REFERENCE TO RELATED APPLICATIONS

This application claims the benefit of priority under 35 U.S.C. §119 of U.S. Provisional Application Ser. No. 61/617,202 filed on Mar. 29, 2012 the content of which is relied upon and incorporated herein by reference in its entirety.

### FIELD

Described herein are compositions including heterocyclic organic compounds. More specifically, described herein are fused heterocyclic naphthalene compounds, methods for making them, and uses thereof.

### TECHNICAL BACKGROUND

Highly conjugated organic materials, due to their interesting electronic and optoelectronic properties, are being investigated for use in a variety of applications, including organic semiconductors (OSCs), field effect transistors (FETs), thin-film transistors (TFTs), organic light-emitting diodes (OLEDs), electro-optic (EO) applications, as conductive materials, as two photon mixing materials, as organic semiconductors, and as non-linear optical (NLO) materials.

In particular, OSCs have attracted a great amount of attentions in the research community due to their advantages over inorganic semiconductors such as processing in any form, exhibiting a high mechanical flexibility, producing at low cost, and having a low weight. Polycyclic aromatic compounds, such as oligothiophenes, acenes, rylenes, phthalocyanens, and polythiophene, have been widely studied as semiconductor materials.

Among the organic p-type semiconductors, pentacene exhibits charge mobilities well above 1 cm²/V·s in organic field effect transistor devices. This number has been set up as a bench mark for new small molecule systems in terms of mobility requirements. However, due to the continuing need for improved performance and stability in semiconductor structures, there is an unmet need to develop better performing OSCs that have improved mobility, are structurally stable, and applicable to the large number of potential applications seen in the various high technology markets.

### SUMMARY

Embodiments comprise a rationally designed a family of alkyl-substituted fused naphthalene hetero ring materials. The materials have several advantages in that it is easier to introduce substituents onto the fused rings allowing for significant improvement of the polymerization process and the polymer material processibility; substituents can be introduced to multiple positions which allows for fine tuning material packing behaviors; introduction of substituted pyrrole structures into the substituted naphthalene results in lower reorganization energy and higher mobility; and introduction of β-substituents on five member ring increases the material stability.

A first embodiment comprises a compound of formula:

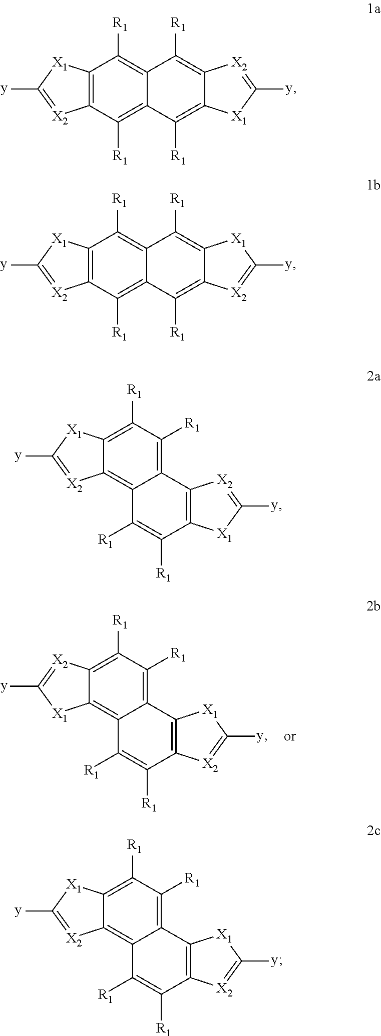

wherein each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; y is H, halo, trialkylsilane, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs; and each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

In some embodiments, the compound is 1a or 1b. In other embodiments, the compound is 2a, 2b, or 2c. In some embodiments, X₁ is NR₁, PR₁, AsR₁, Sb, O, S, Se, or Te; X₂ is N or CR₁; y is H, halo, optionally substituted C₁-C₄₀ alkyl, optionally substituted C₂-C₄₀ alkenyl, optionally substituted C₂-C₄₀ alkynyl, halo, OSO-alkyl, Mg-halo, Zn-halo, Sn(alkyl)₃, B(OH)₂, or B(alkoxy)₂; and each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

In other embodiments, each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, optionally substituted cycloalkyl, optionally substituted cycloalkenyl, halo, optionally substituted heterocyclyl, or an optionally substituted aryl or optionally substituted heteroaryl from the group consisting of phenyl, thiophenyl, furanyl, pyrrolyl, imidazolyl, triazolyl, oxaxolyl, thiazolyl, pyridinyl, pyrimidinyl, triazinyl, naphthalenyl, isoquinolinyl, quinolinyl, or naphthyridinyl.

In some embodiments, for 2a, 2b, or 2c, each X₁ is independently NR₁, PR₁, AsR₁, Sb, O, or Te or Se and each X₂ is independently N, P, As, SiR₁, or CR₁, with the proviso that due to conjugation, X₁ and X₂ may be bonded to one or more additional R₁s. In other embodiments, for 2a, 2b, or 2c, each X₁ is independently NR₁, PR₁, AsR₁, Sb, O, S, Se, or Te, with the proviso that due to conjugation, X₁ may be bonded to one or more additional R₁ and each X₂ is independently N, P, As, or SiR₁, with the proviso that due to conjugation, X₂ may be bonded to one or more additional R₁. In some embodiments, each X₂ is independently N or CR₁, with the proviso that due to conjugation, X₂ may be bonded to one or more additional R₁.

In some embodiments, the compound comprises 1a, 1b, 2a, 2b, or 2c, and the hole reorganization energy is less than 0.35 eV. In some embodiments, the hole reorganization energy is from about 0.05 to about 0.35 eV.

Another embodiment comprises a polymer of formula:

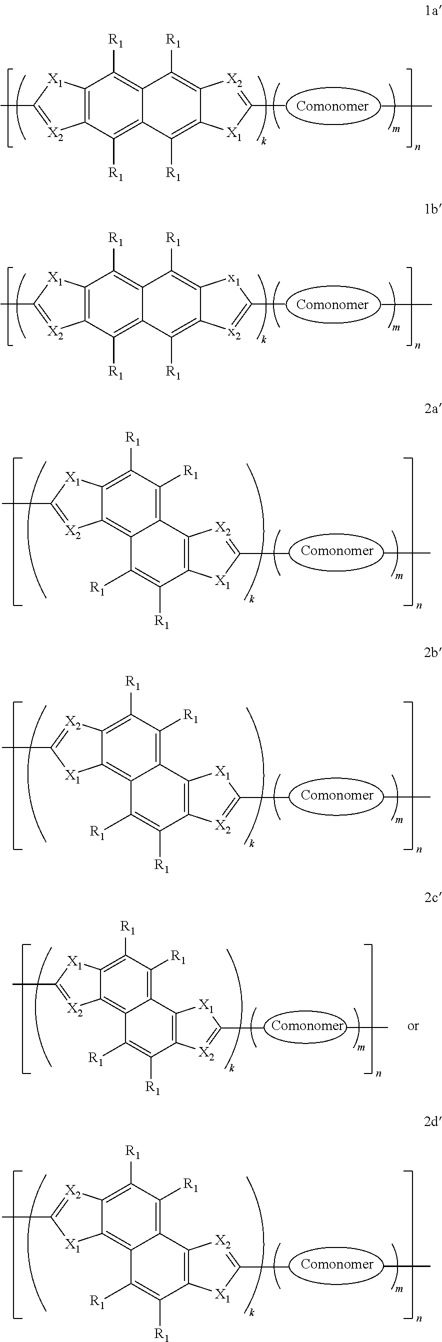

wherein n is an integer greater than zero; k is from 1 to 10; m is from 0 to 10; with the proviso that when m is 0, k is null; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; y is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs; each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; and comonomer comprises an optionally substituted C₂-C₄₀ conjugated alkenyl, optionally substituted C₂-C₄₀ conjugated cycloalkenyl, optionally substitute C₂-C₄₀ conjugated heteroalkenyl, optionally substituted conjugated C₂-C₄₀ hetero cyclo alkenyl, optionally substituted C₆-C₄₀ aryl, optionally substituted C₆-C₄₀ heteroaryl, or:

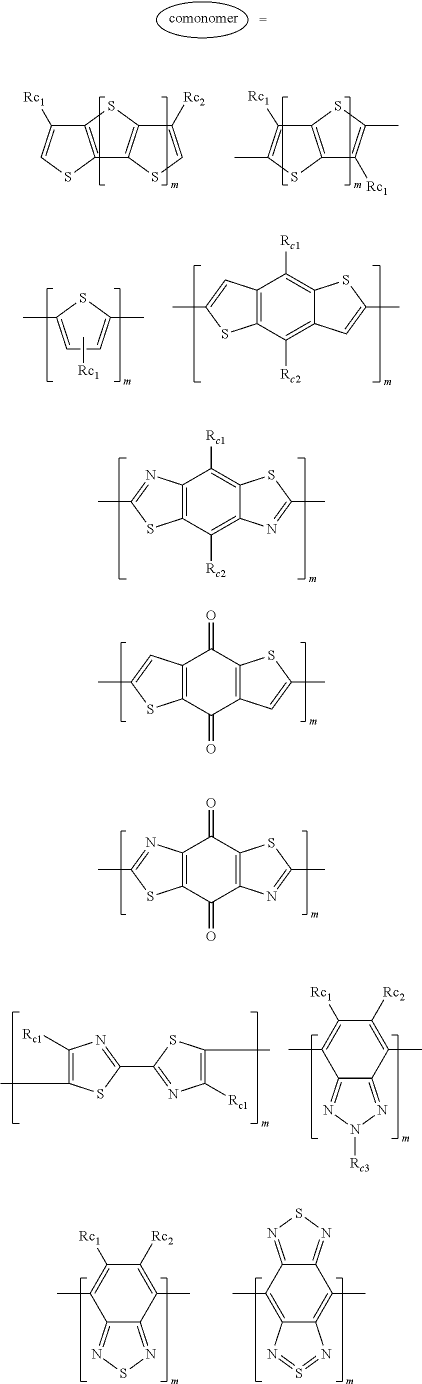

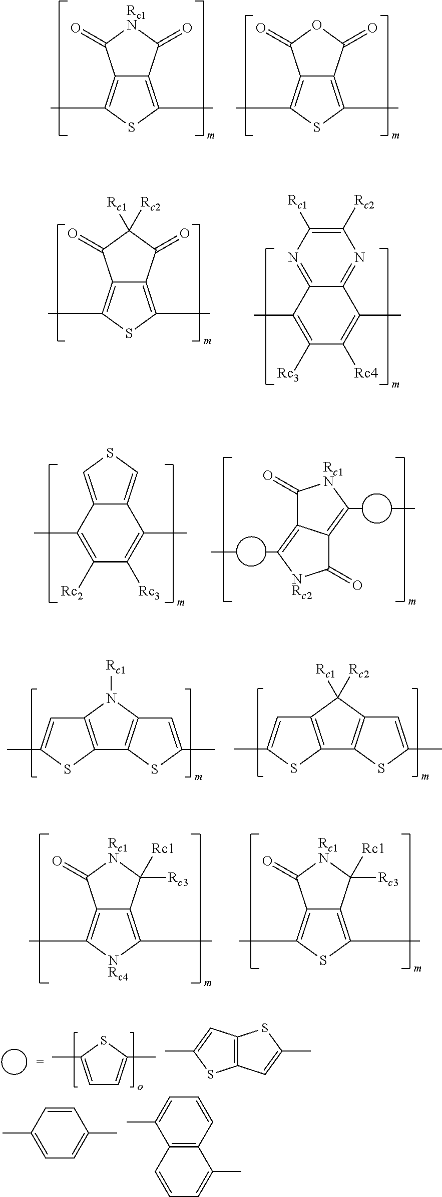

wherein m is 1, 2, or 3; o is 0, 1, 2, or 3; R_{c1}, R_{c2}, R_{c3}, and R_{c4} are independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

In some embodiments, R_{c1}, R_{c2}, R_{c3}, and R_{c4} are independently H, optionally substituted C₁-C₄₀ alkyl, C₂-C₄₀ optionally substituted alkenyl, optionally substituted C₂-C₄₀ alkynyl, optionally substituted cycloalkyl, optionally substituted cycloalkenyl, optionally substituted heterocyclyl, or optionally substituted phenyl, optionally substituted thiophenyl, optionally substituted furanyl, optionally substituted pyrrolyl, optionally substituted imidazolyl, optionally substituted triazolyl, optionally substituted oxaxolyl, optionally substituted thiazolyl, optionally substituted naphthalenyl, optionally substituted isoquinolinyl, optionally substituted quinolinyl, or optionally substituted naphthyridinyl.

In some embodiments, for 1a′, 1b′, 2a′, 2b′, 2c′, or 2d′, X₁ is NR₁, PR₁, AsR₁, Sb, O, S, Se, or Te, with the proviso that due to conjugation, X₁ may be bonded to one or more additional R₁; X₂ is N or CR₁, with the proviso that due to conjugation, X₂ may be bonded to one or more additional R₁; y is H, halo, optionally substituted C₁-C₄₀ alkyl, optionally substituted C₂-C₄₀ alkenyl, optionally substituted C₂-C₄₀ alkynyl, halo, OSO-alkyl, Mg-halo, Zn-halo, Sn(alkyl)₃, B(OH)₂, or B(alkoxy)₂; and each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone. In other embodiments, each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, optionally substituted cycloalkyl, optionally substituted cycloalkenyl, halo, optionally substituted heterocyclyl, or an optionally substituted aryl or optionally substituted heteroaryl from the group consisting of phenyl, thiophenyl, furanyl, pyrrolyl, imidazolyl, triazolyl, oxaxolyl, thiazolyl, pyridinyl, pyrimidinyl, triazinyl, naphthalenyl, isoquinolinyl, quinolinyl, or naphthyridinyl.

In some embodiments, for 1a′, 1b′, 2a′, 2b′, 2c′, or 2d′, n is from 1 to 500; k is from 1-10; and m is from 0-10; with the proviso that if m is 0, then k is null. In some embodiments, the ratio of compound 1a, 1b, 2a, 2b, or 2c to comonomer is from about 10:1 to 1:10.

Another embodiment comprises a method of synthesizing a compound comprising:

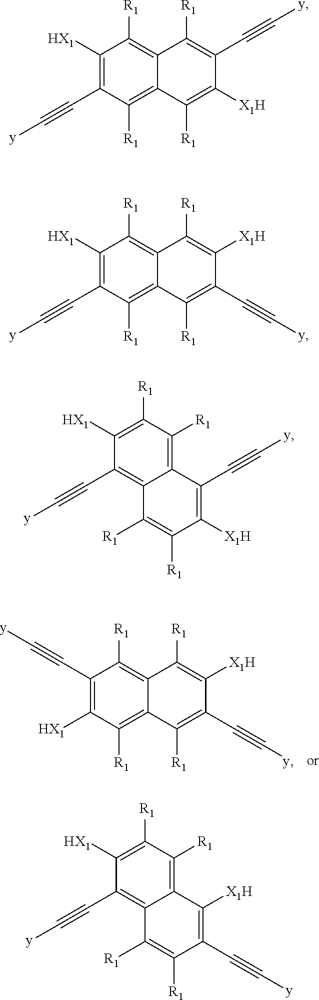

comprising respectively allowing a compound of structure:

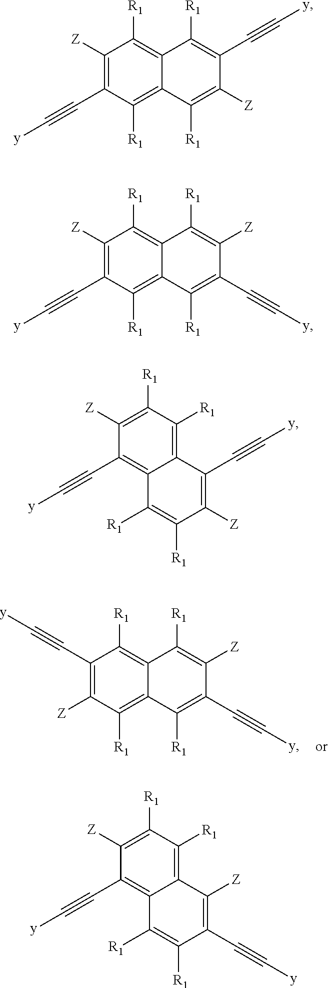

to undergo a substation reaction; wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each X₁ is independently NR₁, PR₁, AsR₁, Sb, O, S, or Se; and each Z is independently Z₁ or I, each Z₁ is independently Cl or Br, y is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

Another embodiment comprises a method of making a compound of structure:

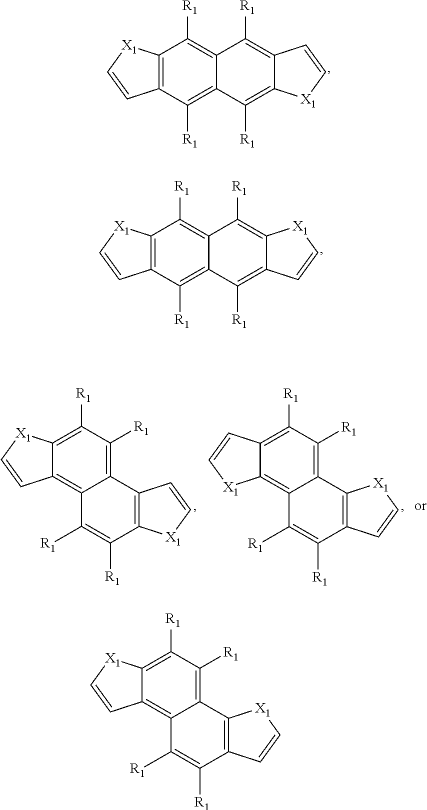

comprising respectively allowing a compound of structure:

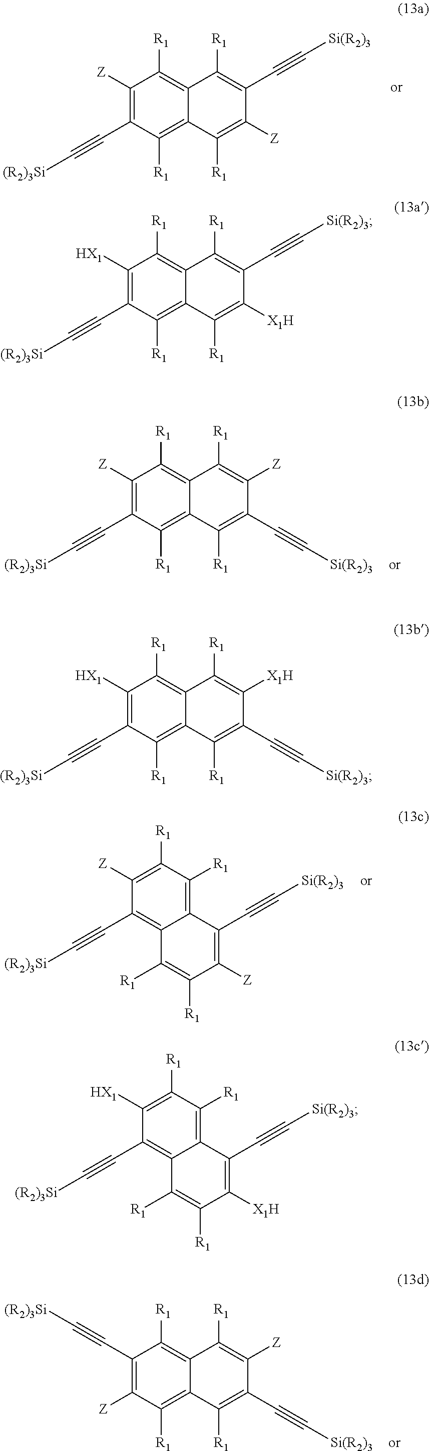

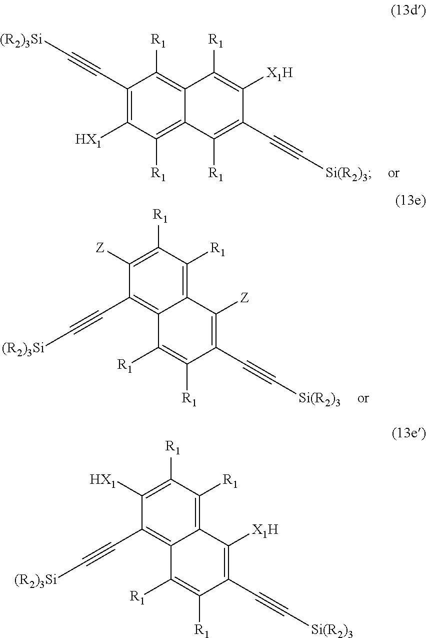

undergo a ring cyclization reaction; wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, or Se, with the proviso that due to conjugation, X₁ may be bonded to one or more additional R₁; and each Z is independently Z₁ or I, each Z₁ is independently Cl or Br.

Another embodiment comprises a method of making a compound of structure:

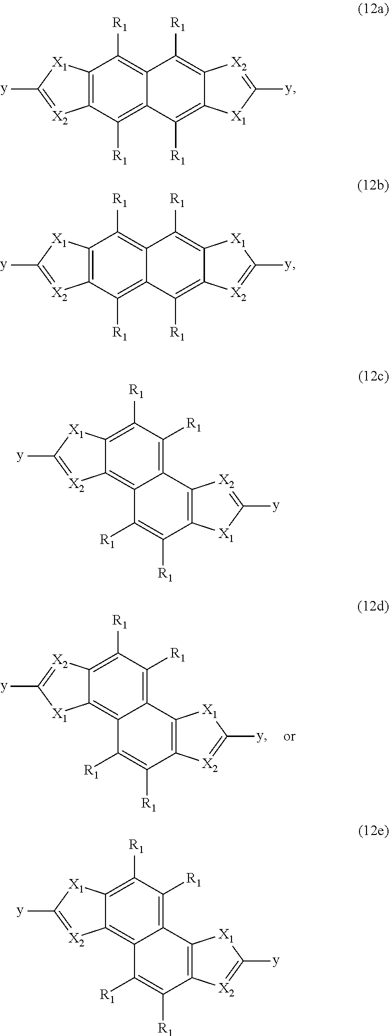

comprising respectively allowing a compound of structure

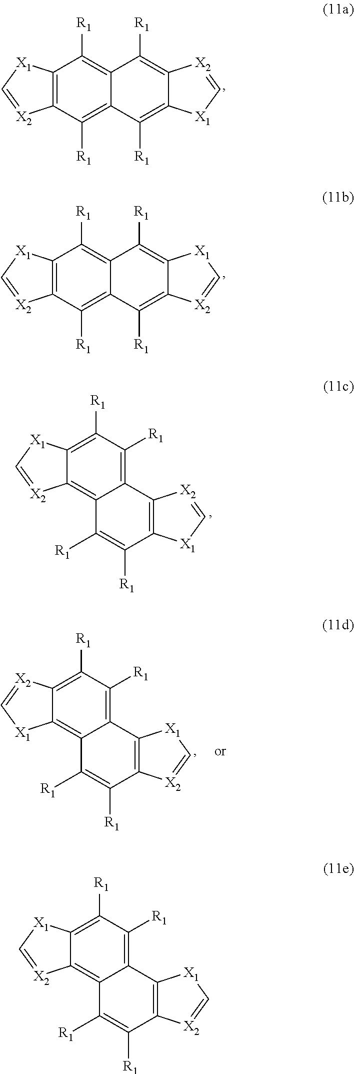

undergo a substitution reaction, wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each X₁ is independently NR₁, PR₁, AsR₁, Sb, O, S, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently Cl or Br; and each y is H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

Another embodiment comprises a method of making a polymer of structure:

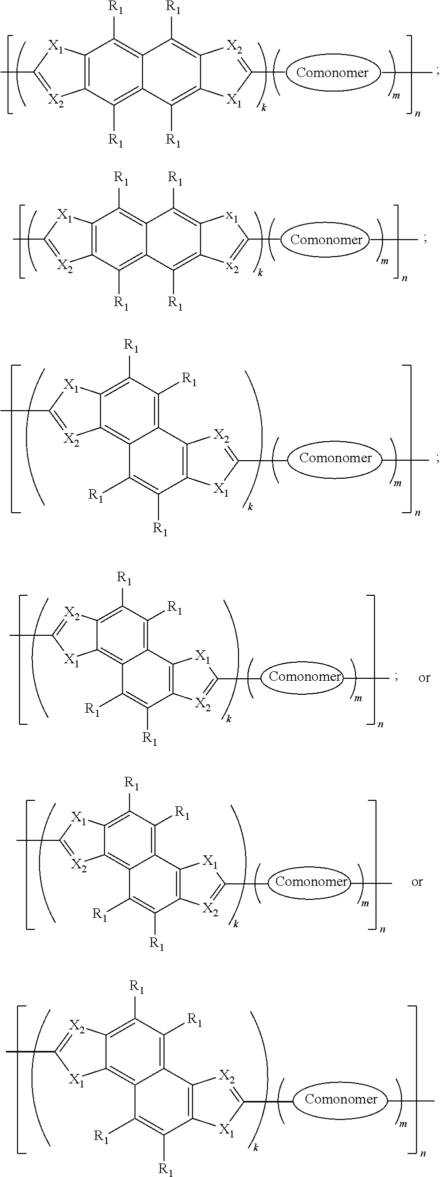

comprising respectively polymerizing a compound of structure:

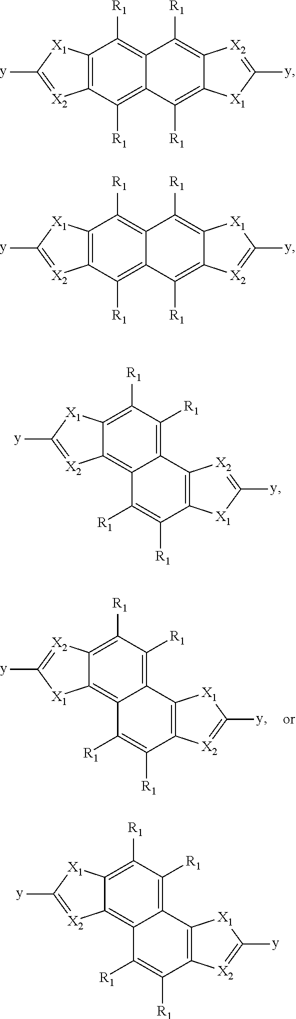

with a compound of structure:

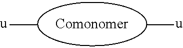

wherein each y is independently H, halo, OSO-alkyl, Mg-halo, Zn-halo, Sn(alkyl)₃, B(OH)₂, B(alkoxy)₂, OTs, or OTf; each u is independently H, halo, OSO-alkyl, Mg-halo, Zn-halo, Sn(alkyl)₃, B(OH)₂, B(alkoxy)₂, OTs, or OTf; each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; wherein comonomer comprises an optionally substituted C₂-C₄₀ conjugated alkenyl, optionally substituted C₂-C₄₀ conjugated cycloalkenyl, optionally substitute C₂-C₄₀ conjugated heteroalkenyl, optionally substituted conjugated C₂-C₄₀ heterocycloalkenyl, optionally substituted C₆-C₄₀ aryl, optionally substituted C₆-C₄₀ heteroaryl, or:

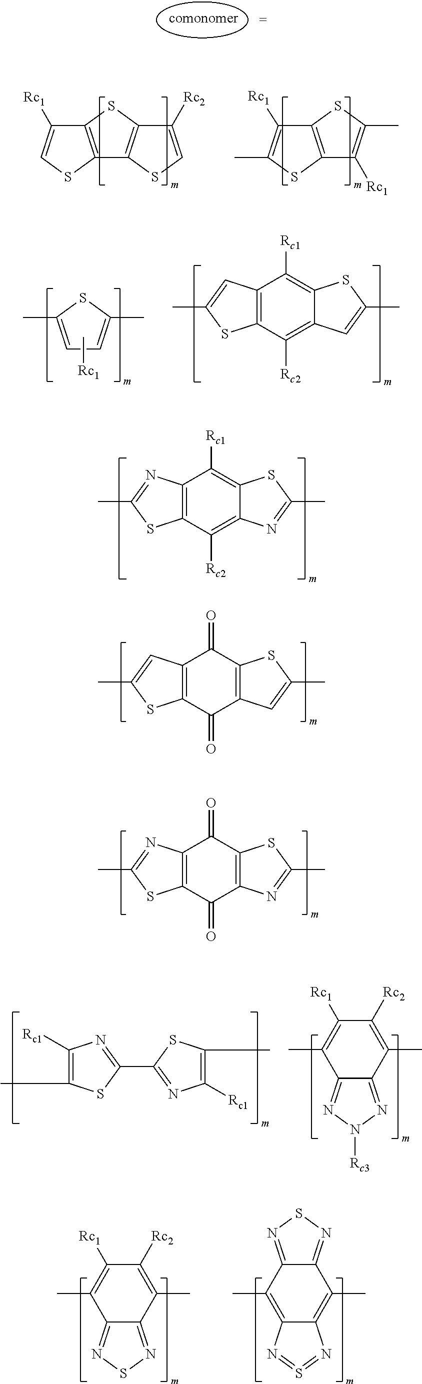

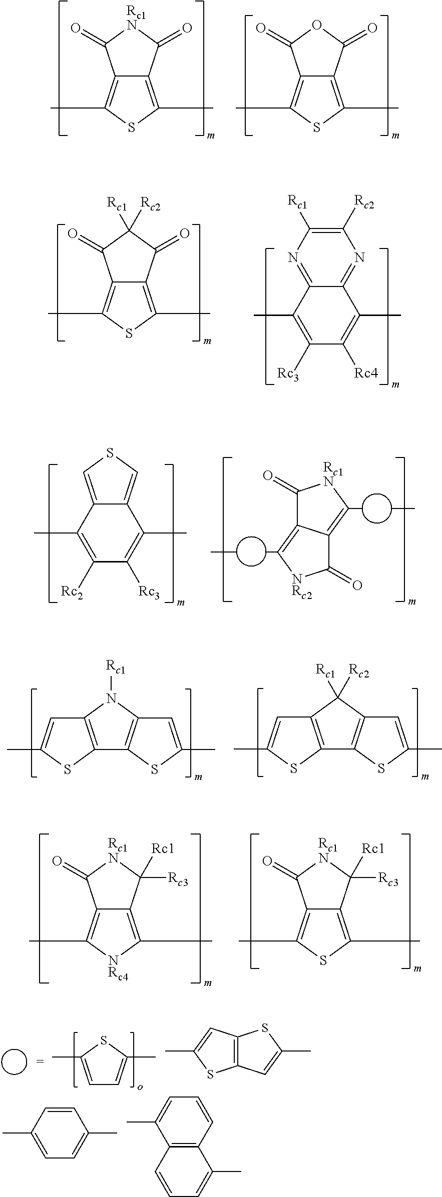

wherein each m is independently 1, 2, or 3; o is 0, 1, 2, or 3; u is R_{c1}, R_{c2}, R_{c3}, and R_{c4} are independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

Another embodiment comprises a device comprising compound 1a, 1b, 2a, 2b, or 2c. Another embodiment comprises a device comprising polymer 1a′, 1b′, 2a′, 2b′, 2c′ or 2d′.

Additional features and advantages will be set forth in the detailed description which follows, and in part will be readily apparent to those skilled in the art from the description or recognized by practicing the embodiments as described in the written description and claims hereof, as well as in the appended drawings.

It is to be understood that both the foregoing general description and the following detailed description are merely exemplary, and are intended to provide an overview or framework for understanding.

### BRIEF DESCRIPTION OF THE DRAWINGS

The accompanying drawings are included to provide a further understanding, and are incorporated in and constitute a part of this specification.

FIG. 1 shows the importance the reorganization energy (R.E.) and the transfer integral in the charge carrier mobility (M). Based on the various plots shown for transfer integrals from 0.4 to 2.0 eV, it is clear that large increases in the transfer integral do not yield significant variation in the mobility, unless the reorganization energies are small.

FIG. 2 is a schematic diagram showing the internal reorganization energy (E) as a function of nuclear configuration (N.C.) for hole transfer from the neutral ground state (100) to a cationic state (200). The figure shows that E varies as a function of various internal reorganization components, λ=λ₀+λ₊, and the ionization potential (I.P.), IP=E₊*−E.

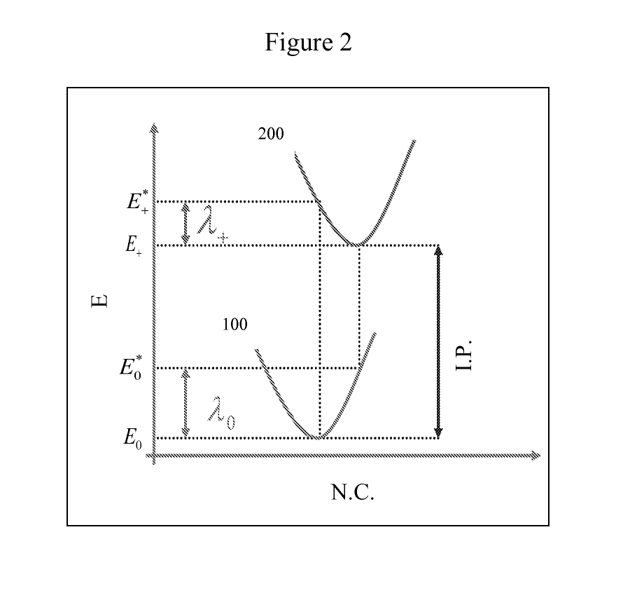

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

The term “alkyl” refers to a monoradical branched or unbranched saturated hydrocarbon chain having 1, 2, 3, 4, 5, 6, 7, 8, 9, 10, 11, 12, 13, 14, 15, 16, 17, 18, 19 or 20 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to: (1) an alkyl group as defined above, having 1, 2, 3, 4 or 5 substituents, typically 1 to 3 substituents, selected from the group consisting of alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2; or (2) an alkyl group as defined above that is interrupted by 1-10 atoms independently chosen from oxygen, sulfur and NRₐ, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR_{SO}, in which R_{SO} is alkyl, aryl, or heteroaryl and n is 0, 1 or 2; or (3) an alkyl group as defined above that has both 1, 2, 3, 4 or 5 substituents as defined above and is also interrupted by 1-10 atoms as defined above.

The term “alkylene” refers to a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, typically 1-10 carbon atoms, more typically 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” refers to: (1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)—R_{SO}, where R_{SO} is alkyl, aryl, or heteroaryl and n is 0, 1 or 2; or (2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocyclyl, or groups selected from carbonyl, carboxyester, carboxyamide and sulfonyl; or (3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), and the like.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “alkoxy” refers to the group R—O—, where R is an optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio” refers to the group R_{S}—S—, where R_{S} is as defined for alkoxy.

The term “alkenyl” refers to a monoradical of a branched or unbranched unsaturated hydrocarbon group typically having from 2 to 20 carbon atoms, more typically 2 to 10 carbon atoms and even more typically 2 to 6 carbon atoms and having 1-6, typically 1, double bond (vinyl). Typical alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. In the event that alkenyl is attached to nitrogen, the double bond cannot be alpha to the nitrogen.

The term “substituted alkenyl” refers to an alkenyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “alkynyl” refers to a monoradical of an unsaturated hydrocarbon, typically having from 2 to 20 carbon atoms, more typically 2 to 10 carbon atoms and even more typically 2 to 6 carbon atoms and having at least 1 and typically from 1-6 sites of acetylene (triple bond) unsaturation. Typical alkynyl groups include ethynyl, (—CCH), propargyl (or prop-1-yn-3-yl, —CH₂CCH), and the like. In the event that alkynyl is attached to nitrogen, the triple bond cannot be alpha to the nitrogen.

The term “substituted alkynyl” refers to an alkynyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “aminocarbonyl” refers to the group —C(O)NR_{N}R_{N} where each R_{N} is independently hydrogen, alkyl, aryl, heteroaryl, heterocyclyl or where both R_{N} groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acylamino” refers to the group —NR_{NCO}C(O)R where each R_{NCO} is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “aryl” refers to an aromatic carbocyclic group of 6 to 20 carbon atoms having a single ring (e.g., phenyl) or multiple rings (e.g., biphenyl), or multiple condensed (fused) rings (e.g., naphthyl or anthryl). Typical aryls include phenyl, naphthyl and the like.

Unless otherwise constrained by the definition for the aryl substituent, such aryl groups can optionally be substituted with from 1 to 5 substituents, typically 1 to 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “aryloxy” refers to the group aryl-O— wherein the aryl group is as defined above, and includes optionally substituted aryl groups as also defined above. The term “arylthio” refers to the group aryl-S—, where aryl is as defined as above.

The term “amino” refers to the group —NH₂.

The term “substituted amino” refers to the group —NR_{w}R_{w} where each R_{w} is independently selected from the group consisting of hydrogen, alkyl, cycloalkyl, carboxyalkyl (for example, benzyloxycarbonyl), aryl, heteroaryl and heterocyclyl provided that both R_{w} groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “carboxyalkyl” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, and —S(O)ₙR_{SO}, in which R_{SO} is alkyl, aryl, or heteroaryl and n is 0, 1 or 2.

The term “cycloalkyl” refers to carbocyclic groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like.

The term “cycloalkenyl” refers to carbocyclic groups of from 3 to 20 carbon atoms having a single cyclic ring or multiple condensed rings with at least one double bond in the ring structure.

The terms “substituted cycloalkyl” or “substituted cycloalkenyl” refer to cycloalkyl or cycloalkenyl groups having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “halogen” or “halo” refers to fluoro, bromo, chloro, and iodo.

The term “acyl” denotes a group —C(O)R_{CO}, in which R_{CO} is hydrogen, optionally substituted alkyl, optionally substituted cycloalkyl, optionally substituted heterocyclyl, optionally substituted aryl, and optionally substituted heteroaryl.

The term “heteroaryl” refers to a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen and sulfur within at least one ring. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, triazole, oxazole, thiazole, naphthyridine, and the like as well as N-oxide and N-alkoxy derivatives of nitrogen containing heteroaryl compounds, for example pyridine-N-oxide derivatives.

Unless otherwise constrained by the definition for the heteroaryl substituent, such heteroaryl groups can be optionally substituted with 1 to 5 substituents, typically 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “heteroaryloxy” refers to the group heteroaryl-O—.

The term “heterocyclyl” refers to a monoradical saturated or partially unsaturated group having a single ring or multiple condensed rings, having from 1 to 40 carbon atoms and from 1 to 10 hetero atoms, typically 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclyl substituent, such heterocyclyl groups can be optionally substituted with 1, 2, 3, 4 or 5, and typically 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “thiol” refers to the group —SH.

The term “substituted alkylthio” refers to the group —S— substituted alkyl.

The term “heteroarylthiol” refers to the group —S-heteroaryl wherein the heteroaryl group is as defined above including optionally substituted heteroaryl groups as also defined above.

The term “sulfoxide” refers to a group —S(O)R_{SO}, in which R_{SO} is alkyl, aryl, or heteroaryl. “Substituted sulfoxide” refers to a group —S(O)R_{SO}, in which R_{SO} is substituted alkyl, substituted aryl, or substituted heteroaryl, as defined herein.

The term “sulfone” refers to a group —S(O)₂R_{SO}, in which R_{SO} is alkyl, aryl, or heteroaryl. “Substituted sulfone” refers to a group —S(O)₂R_{SO}, in which R_{SO} is substituted alkyl, substituted aryl, or substituted heteroaryl, as defined herein.

The term “keto” refers to a group —C(O)—.

The term “thiocarbonyl” refers to a group —C(S)—.

The term “carboxy” refers to a group —C(O)OH.

The term “conjugated group” is defined as a linear, branched or cyclic group, or combination thereof, in which p-orbitals of the atoms within the group are connected via delocalization of electrons and wherein the structure can be described as containing alternating single and double or triple bonds and may further contain lone pairs, radicals, or carbenium ions. Conjugated cyclic groups may comprise both aromatic and non-aromatic groups, and may comprise polycyclic or heterocyclic groups, such as diketopyrrolopyrrole. Ideally, conjugated groups are bound in such a way as to continue the conjugation between the thiophene moieties they connect.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Embodiments comprise a rationally designed a family of alkyl-substituted fused naphthalene hetero ring materials. The materials have several advantages in that it is easier to introduce substituents onto the fused rings allowing for significant improvement of the polymerization process and the polymer material processibility; substituents can be introduced to multiple positions which allows for fine tuning material packing behaviors; introduction of substituted pyrrole structures into the substituted naphthalene results in lower reorganization energy and higher mobility; and introduction of β-substituents on five member ring increases the material stability.

In one aspect, described herein are compositions comprising the formula 1a, 1b, 2a, 2b, or 2c:

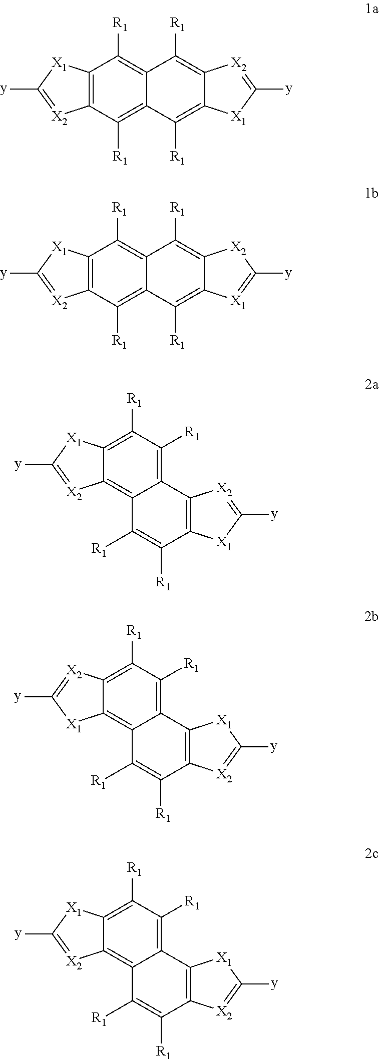

wherein each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁ with the proviso that due to conjugation, X₂ may be bonded to one or more additional R₁; y is H, halo, trialkylsilane, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

Each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted hetero aralkyl, heteroaryloxy, optionally substituted heterocyclyl, thiol, alkylthio, heteroarylthiol, optionally substituted sulfoxide, or optionally substituted sulfone.

In some embodiments, y is H, halo, —OSO-alkyl, —Mg-halo, —Zn-halo, —Sn(alkyl)₃, —B(OH)₂, or —B(alkoxy)₂. In some embodiments, each R₁ is independently H, optionally substituted alkyl, optionally substituted alkenyl, optionally substituted alkynyl, optionally substituted aryl, optionally substituted heteroaryl, optionally substituted heteroaralkyl, optionally substituted cycloalkyl, optionally substituted cycloalkenyl, optionally substituted heterocyclyl, or aralkyl. In some embodiments, each R₁ is independently H, optionally substituted alkyl, optionally substituted alkenyl, optionally substituted alkynyl, optionally substituted cycloalkyl, optionally substituted cycloalkenyl, optionally substituted heterocyclyl, or optionally substituted phenyl, optionally substituted thiophenyl, optionally substituted furanyl, optionally substituted pyrrolyl, optionally substituted imidazolyl, optionally substituted triazolyl, optionally substituted oxaxolyl, optionally substituted thiazolyl, optionally substituted napthalenyl, optionally substituted isoquinolinyl, optionally substituted quinolinyl, or optionally substituted naphthyridinyl.

In some embodiments, for 2a, 2b, or 2c, each X₁ is independently NR₁, PR₁, AsR₁, Sb, O, or Te, with the proviso that due to conjugation, X₁ may be bonded to one or more additional R₁; and each X₂ is independently N, P, As, SiR₁, or CR₁, with the proviso that due to conjugation, X₂ may be bonded to one or more additional R₁. In other embodiments, for 2a, 2b, or 2c, each X₁ is independently NR₁, PR₁, AsR₁, Sb, O, S, Se, or Te, with the proviso that due to conjugation, X₁ may be bonded to one or more additional R₁ and each X₂ is independently N, P, As, or SiR₁, with the proviso that due to conjugation, X₂ may be bonded to one or more additional R₁.

In another aspect, the composition comprises formula 1a′, 1b′, 2a′, 2b′, 2c′, or 2d′:

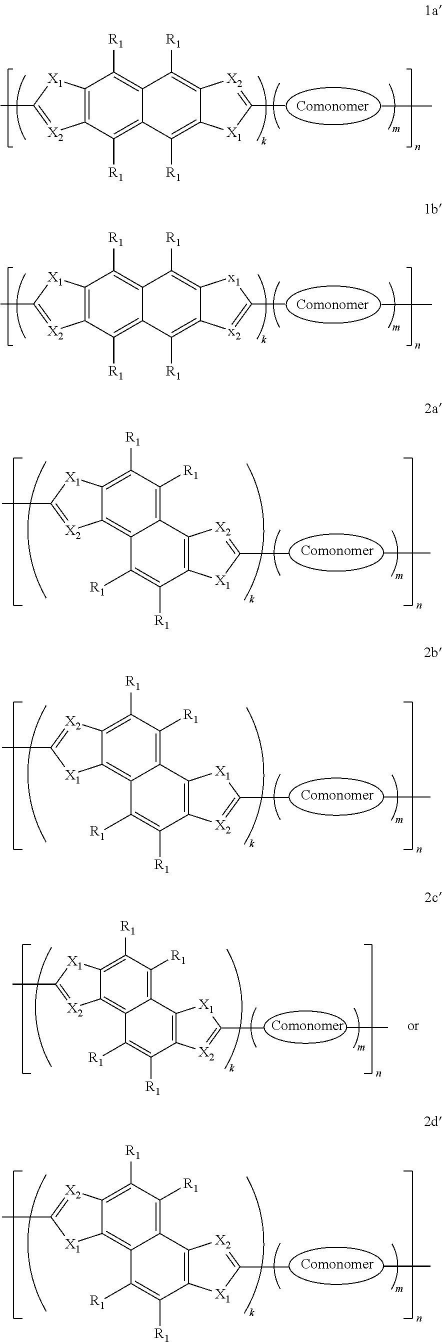

wherein n is an integer greater than zero; X₁, X₂, y, and R₁ all have the same meanings as above; k is from 1 to 10 with the proviso that when m is 0 (meaning no comonomer is present), k is null (meaning that the “k” term vanishes as it would become equivalent to the “n” term—therefore the polymer comprises “n” fused heterocyclic naphthalene groups as described by 1a′, 1b′, 2a′, 2b′, 2c′, or 2d′); m is from 0 to 10; the ratio of k to m may be from 1:10 to 10:1 with the exception that when m is 0 the ratio of k to m is null; and n is from about 1 to 500. In some embodiments, k is 1, 2 or 3. In some embodiments, m is 1, 2, or 3. In some embodiments, the ratio of k to m is from about 3:1 to about 1:3. In some embodiments, n is from about 3 to about 20, about 3 to about 15, about 3 to about 12, about 3 to about 10, or about 5 to about 9. In some embodiments, n is about 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, or 500.

Comonomer, as used herein, describes a conjugated system such as any aromatic structure, double or triple bonds, or conjugated structures. Examples of comonomers include, but are not limited to:

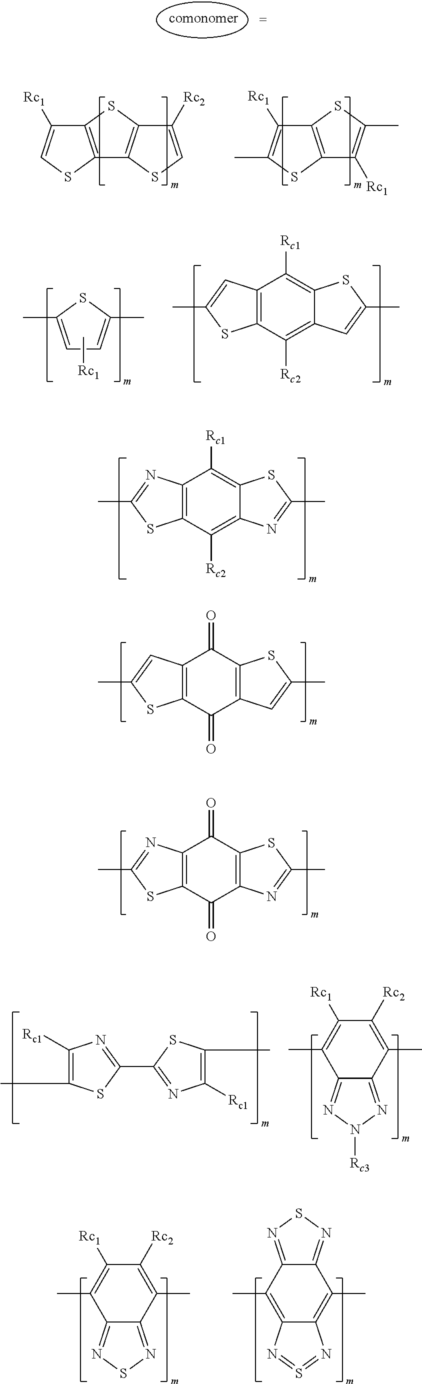

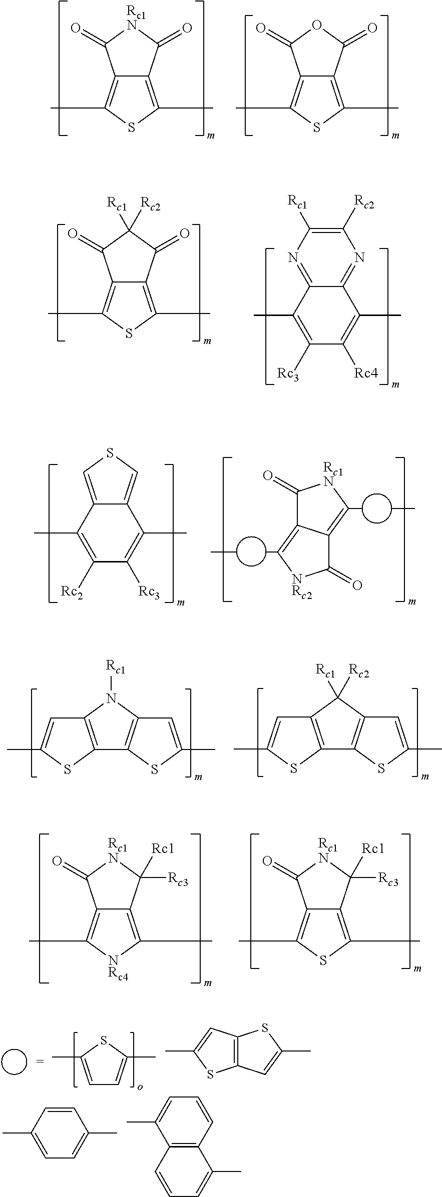

wherein each m is independently is 1, 2, or 3; o is 0, 1, 2, or 3; R_{c1}, R_{c2}, R_{c3}, and R_{c4} are independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone. In some embodiments, R_{c1}, R_{c2}, R_{c3}, and R_{c4} are independently H, optionally substituted alkyl, optionally substituted alkenyl, optionally substituted alkynyl, optionally substituted aryl, optionally substituted heteroaryl, optionally substituted heteroaralkyl, optionally substituted cycloalkyl, optionally substituted cycloalkenyl, optionally substituted heterocyclyl, or aralkyl. In some embodiments, R_{c1}, R_{c2}, R_{c3}, and R_{c4} are independently H, optionally substituted alkyl, optionally substituted alkenyl, optionally substituted alkynyl, optionally substituted cycloalkyl, optionally substituted cycloalkenyl, optionally substituted heterocyclyl, or optionally substituted phenyl, optionally substituted thiophenyl, optionally substituted furanyl, optionally substituted pyrrolyl, optionally substituted imidazolyl, optionally substituted triazolyl, optionally substituted oxaxolyl, optionally substituted thiazolyl, optionally substituted napthalenyl, optionally substituted isoquinolinyl, optionally substituted quinolinyl, or optionally substituted naphthyridinyl.

In another aspect, embodiments may be produced through a series of synthetic steps. For illustrative purposes, reaction Schemes 1-7 depicted below provide potential routes for synthesizing the embodiments as well as key intermediates. The methods disclosed in the instant Schemes and Examples are intended for purposes of exemplifying only and are not to be construed as limitations thereon.

Those skilled in the art will appreciate that other synthetic routes may be used to synthesize the inventive compounds. Some aspects of some embodiments may be synthesized by synthetic routes that include processes analogous to those well-known in the chemical arts, particularly in light of the description contained herein. Although specific starting materials and reagents are depicted in the schemes and discussed below, other starting materials and reagents can be easily substituted to provide a variety of derivatives and/or reaction conditions. The starting materials are generally available from commercial sources, such as Aldrich Chemicals (Milwaukee, Wis.), or are readily prepared using methods well known to those skilled in the art (e.g., prepared by methods generally described in Louis F. Fieser and Mary Fieser, REAGENTS FOR ORGANIC SYNTHESIS, v. 1-19, Wiley, New York (1967-1999 ed.), or BEILSTEINS HANDBUCH DER ORGANISCHEN CHEMIE, 4, Aufl. ed. Springer-Verlag, Berlin, including supplements (also available via the Beilstein online database)). In addition, many of the compounds prepared by the methods described below can be further modified in light of this disclosure using conventional chemistry well known to those skilled in the art.

In another aspect, compounds comprising 1a, 1b, 2a, 2b, or 2c and intermediates leading to 1a, 1b, 2a, 2b, or 2c may be synthesized. Scheme 1 exemplifies one possible synthetic routes for forming embodied compounds 1a and 1b:

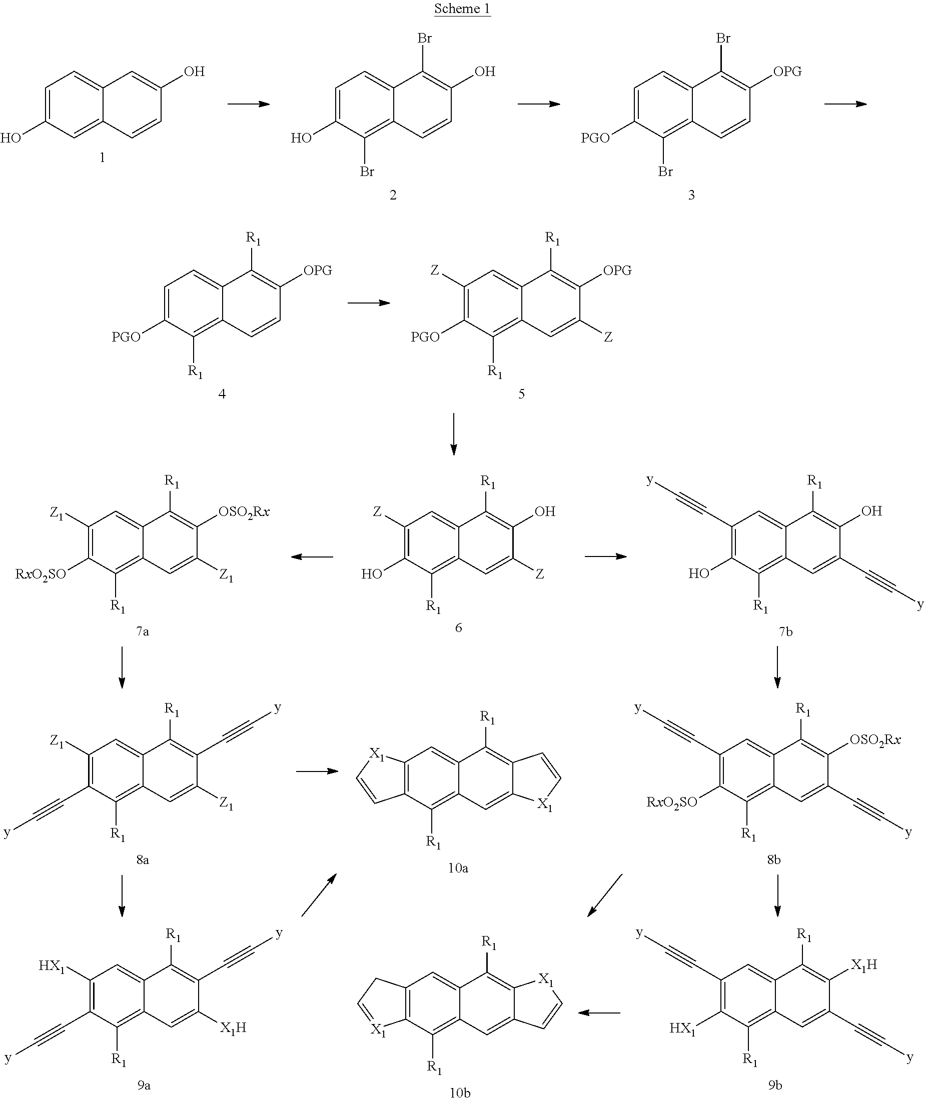

wherein y, X₁ and R₁ have the same meanings as above (X₂ is shown as —CH, but could be generalized to any X₂), PG represents a protecting group, such as Me, MOM (methoxymethyl), EOM (ethoxymethyl), MTM (methylthiomethoxy), THP (tetrahydropyranyl), etc., each Z is independently Z₁ or I, each Z₁ is independently Cl or Br, each SO₂Rₓ is independently SO₂(CF₂)ₓF, wherein x is from 1-20, Ts, or Ms.

As noted in Scheme 1, a naphthalene diol compound, (1), may be reacted with N-bromosuccinimide in THF at a 1:2 ratio, and quenched with saturated sodium thiosulfate to produce 1,5-dibromonaphthalene-2,6-diol, (2). Compound (2) may be reacted with an alcohol protecting group, such as excess chloro(methoxy)methane in dichloromethane and diisopropylethylamine, and quenched with water after 22 hours. After extraction, 1-5-dibromo-2,6-bis(PG)naphthalene, (3), may be obtained. Compounds of form (3) may be combined with n-butyl lithium (2.4 equiv) in an organic solvent, then combined with a halo-alkyl, such as iodomethane, in THF, extracted with saturated sodium sulfate, washed, dried, and purified to give 3,7,-dibromo-2,6-bis(PG)-1,5-dialkylnaphthalene, compound (4).

Compounds of form (5) may be formed via halogenation of compound (4). The general reaction for iodination is shown below and is very similar to bromination of (4), except that I₂ could be used instead of CF₂BrCF₂Br:

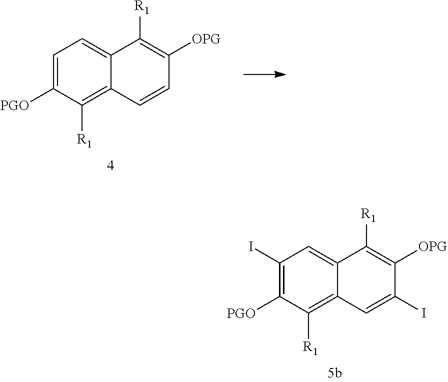

For example, compound (4) may be combined with n-BuLi (2.4 eq.) in solvent (e.g., anhydrous ethyl ether) at room temperature. After sufficient time, the solution can be cooled to 0° C. and a THF solution of diiodine (I₂) added. The resulting mixture is allowed to warm to room temperature over time, quenched, and the aqueous layer extracted. The combined organic extracts can be washed and dried to give compound (5) ((5b) in the case of diiodine). After evaporation, the resulting crude product can be purified by column chromatography on silica gel.

Compounds of structure (6), 3,7-dihalo-1,5-dialkylnaphthalene-2,6-diol, may be produced from compound (5) by combining (5) with 6N HCl in dichloromethane/methanol (1:18 ratio), heating, stirring overnight, pouring into water, and extracting with ethyl acetate.

Compounds of structure (7a), 3,7-Dihalo-1,5-dialkylnaphthalene-2,6-diyl bis(trifluoromethanesulfonate), may be formed by reaction of compound (6) in an organic solvent, such as pyridine and dichloromethane, with trifluoromethanesulfonic anhydride (1:2), mixed with water and 1M HCl, extracted with dichloromethane and concentrated in vacuo. The residue may then be purified to give compound (7a) at about 80% yield.

Compounds (7b) may be formed from compounds (6) by adding Tetrakis(triphenylphosphine)palladium(0) ((Pd(PPh3)4), CuI, triethylamine, diisopropylamine and terminal alkynes to a degassed solution of (6), stirring at 80° C., and adding water and 1M HCl after approximately 15 minutes. The resulting mixture can be extracted and the combined organic layers dried and concentrated to give (7b) (see, e.g., Zhao, Y.; et al. 15 CHEM. EUR. J. 13356 (2009)), incorporated by reference in its entirety).

Compounds (8a) may be formed from compounds (7a) by adding bis(triphenyphosphine) palladium chloride ((Pd(PPh₃)₂Cl₂), CuI, and terminal alkynes to a degassed solution of (7a) in solvent (e.g., THF or DMF), stirring at room temperature, and adding water and 1M HCl after approximately 1 hour. The resulting mixture can be extracted and the combined organic layers dried and concentrated to give (8a) (see, e.g. Shinamura, S. et al. 133 J. AM. CHEM. SOC. 5024 (2011), incorporated by reference in its entirety).

Compounds of structures (8b) may be formed by reaction of compound (7b) in an organic solvent, such as pyridine and dichloromethane, with trifluoromethanesulfonic anhydride (1:2), mixed with water and 1M HCl, extracted with dichloromethane and concentrated in vacuo. The residue may then be purified to give compound (8b) (see, e.g. Shinamura, S. et al. 133 J. AM. CHEM. SOC. 5024 (2011), incorporated by reference in its entirety).

When Z₁ is Br, Compound (9a) may be formed from compound (8a) via reaction of (8a) with tBuONa, tris(dibenzylideneacetone)dipalladium(0), and 2,2′-bis(diphenylphosphino)-1,1′-binaphthyl in dry solvent. Primary amines may be added via a syringe and the mixture was refluxed under nitrogen for 4 h. After cooling to room temperature, water can be added to the solution and the reaction mixture extracted. After drying and solvent evaporation, the residue may be purified to give compound (9a) (see, e.g., Lu et al., 160 SYN. METALS 1438-41 (2010), incorporated by reference in its entirety).

In the case wherein x is NHR and Z₁ is Cl, compound (9a) may be formed from compound (8a) via combination with aryl chloride, amine, KOtBu and a catalyst in 1,2-dimethoxyethane. The mixture may be stirred at room temperature in an air atmosphere and monitored by GC/GC-MS. The reaction may be quenched with water, extracted with solvent, dried, concentrated and purified to give the desired product (see, e.g., Lee et al., 13 ORG. LETT. 5540 (2011), incorporated by reference in its entirety).

Compound (9b) may be formed from compound (8b) via reaction of (8b) with Cs₂CO₃, Tris(dibenzylideneacetone)dipalladium(0), and 2,2′-bis(diphenylphosphino)-1,1′-binaphthyl in dry solvent. Primary amines may be added via a syringe and the mixture was refluxed under nitrogen for 4 h. After cooling to room temperature, water can be added to the solution and the reaction mixture extracted. After drying and solvent evaporation, the residue may be purified to give compound (9b).

Alternatively, compound (9b) may be formed from compound (8b) via reaction of (8b) with Si(SH)(i-Pr)₃ in solvent (Thompson et al., 21 BIOORG. MED. CHEM. LETT. 3764-66 (2011), herein incorporated by reference), as noted below:

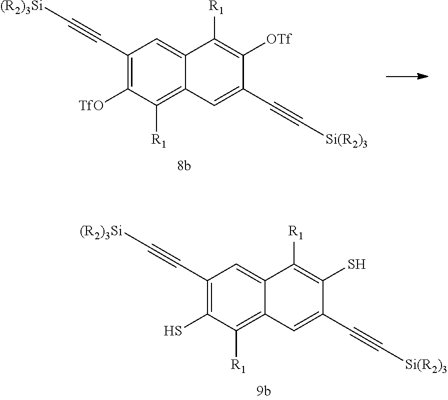

wherein R₂ is an alkyl or aryl.

Compounds (10a) and (10b) may be formed from compounds (9a) and (9b), respectively, via a number of ring cyclization processes. The first comprises combining in a dried pressure tube either compound (9a) or (9b) in DMSO or an alternative solvent with finely crushed KOH. The resulting reaction mixture was heated at 120° C. for 17 hours and was extracted with ethyl acetate, dried, concentrated and purified by column chromatography (see, e.g., Verma et al., 13 ORG. LETT. (2011), incorporated by reference in its entirety). The second comprises combining (9a) or (9b) with a ruthenium catalyst in dry/deoxygenated solvent (e.g., THF) in a scintillation vial under an inert atmosphere, such as in a glove box. The mixture can then be sealed and heated (˜70° C.) for an extended period of time (˜2 days), while being monitored for completion of the reaction. The resulting products may be purified by column chromatography (see, e.g., Nair et al., 16 CHEM. EUR. J. 7992 (2010), incorporated by reference in its entirety). A third possible method involves (9a) or (9b) with triethylamine and CuI in a solvent (e.g., DMF) under an inert atmosphere (e.g., a Schlenk line). The flask may then be sealed and heated with reaction progress monitored by gas chromatography. The reaction products may be purified, for example, by adsorbing directly onto a Teledyne Isco silica load cartridge followed by elution onto a Teledyne Isco column using a 0 to 20% ethyl alcohol/hexane solution (see, e.g., Arnold et al., 13 ORG. LETT. 5576 (2011), incorporated by reference in its entirety). Another method of forming compounds (10a) and (10b) from compounds (9a) and (9b) comprises reacting (9b) or (9a) with NaOH in ethyl acetate and N-methylpyrrolidone at 5C, then allowing the reaction to warm to room temperature for 30 minutes (WO 2011147690, herein incorporated by reference in its entirety).

Alternatively, Compounds (10a) and (10b) may be formed from compounds (8a) and (8b), respectively, using a number of different methods. The first is the general cyclization procedure for dibromodiethynylnaphthalene analogues described in Shoji et al., 133 J. AMER. CHEM Soc. 5024-5035 (2011) (incorporated by reference in its entirety). The procedure combines Na₂S in NMP with (8a) or (8b) and heating to about 185° C. for about 12 hours, then adding the solution to a saturated aqueous ammonium chloride solution to precipitate. The precipitate is collected by filtration, washed, and purified by vacuum sublimation to give (10a) or (10b). Second, per Guilarte et al., 13 ORG. LETT. 5100-5103 (2011) (incorporated by reference in its entirety), a solution of (8a) or (8b) is combined with Pd₂DBA₃, LiHMDS, and 4,5-bis(diphenylphosphino)-9,9-dimethylxanthene (Xantphos) in dry solvent (e.g., toluene) and stirred under inert atmosphere for a short time (˜2 min). Then TIPS-SH can be added and the mixture stirred at ˜120° C. until all aryl bromide is consumed as measured by GC-MS. After cooling, TBAF (3 equivalents) can be added to the mixture and the mixture is stirred for 2 hours. The resulting products may be extracted and purified by column chromatography. The third method is similar to the second, in that it combines a solution of (8a) or (8b) is combined with Pd₂DBA₃, LiHMDS, and 4,5-bis(diphenylphosphino)-9,9-dimethylxanthene (Xantphos) in dry solvent (e.g., toluene) and stirred under inert atmosphere for a short time (˜2 min), then addition of TIPS-SH. The resulting solution may be stirred under inert atmosphere in a microwave at 130° C. and 300 W until all aryl bromide is consumed as measured by GC-MS. Then Cs₂CO₃ can be added to the mixture and microwave irradiation continued until the reaction is complete. The resulting products may be extracted and purified by column chromatography (Guilarte et al., 13 ORG. LETT. 5100-5103 (2011) (incorporated by reference in its entirety)).

Schemes 2 and 3 are synthetically similar to Scheme 1, but provide for the synthesis of the 2a, 2b, and 2c:

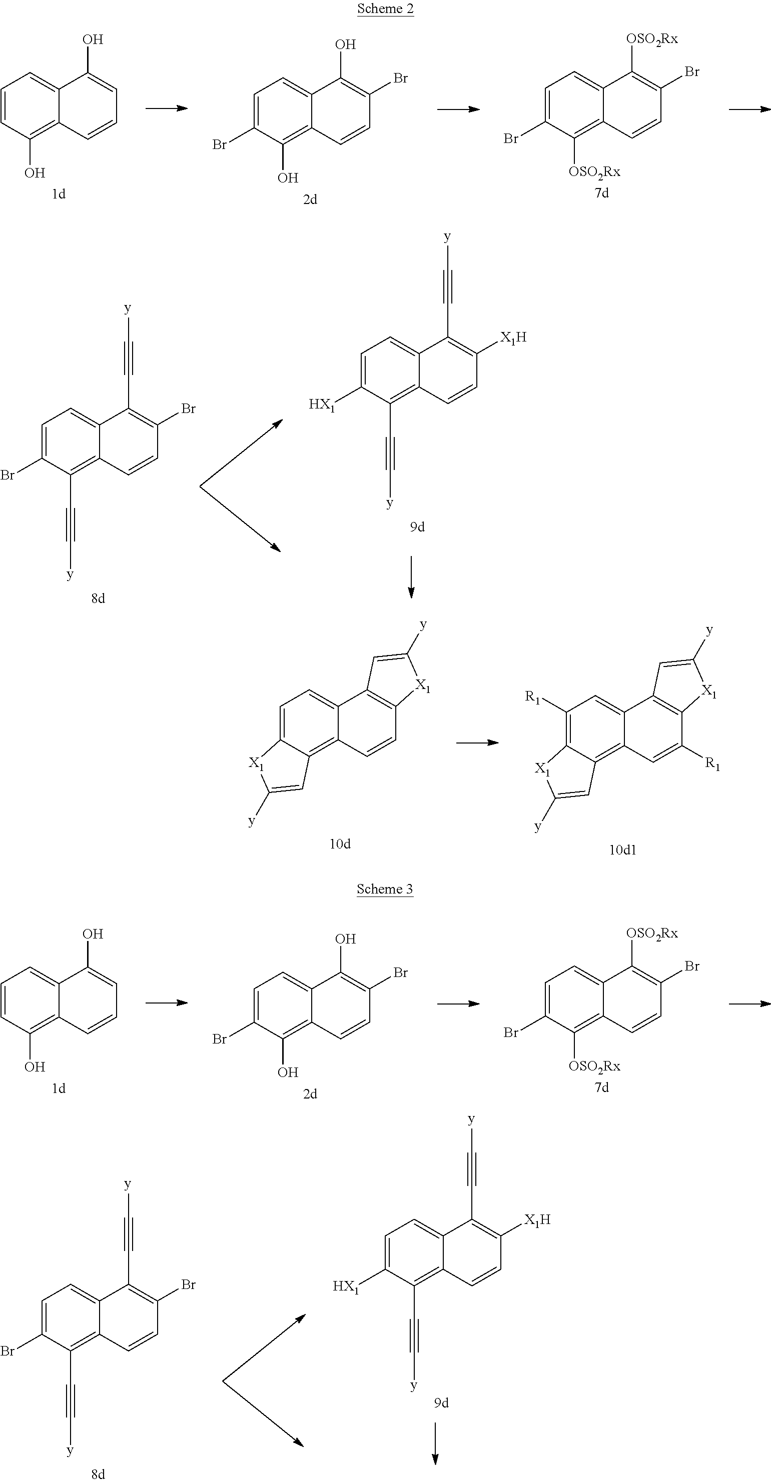

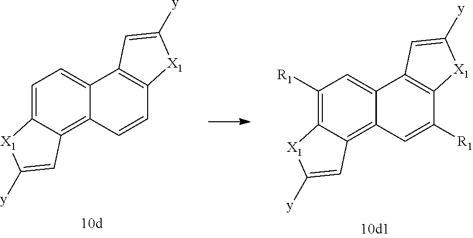

Wherein R₁, y, Z₁, X₁, and Rₓ are the same as described in Scheme 1 (X₂ is shown as —CH, but could be generalized to any X₂). The reaction schemes for Schemes 2 and 3 are somewhat simpler in that the hydroxide group does not need to be protected as no R₁ group is being added as in Scheme 1. Production of compounds (7c)-(10c) and (7d)-(10d) is done as noted above for (7a)-(10a). Compounds of structures (10c1) and (10d1), may be formed by reaction of compound (10c) and (10d) respectively. n-Butyllithium in hexanes (Aldrich) was added dropwise to a solution of (10c) or (10d) respectively in an organic solvent, such as tetrahydrafuran, may afford the lithiation product of (10c) or (10d) respectively, which could be quenched with electrophilic reagents. After workup, the residue may then be purified to give either compound (10c1) or compound (10d1) (see, e.g. Katritzky, A. et al, 53 J. ORG. CHEM. 794 (1988)), incorporated by reference in its entirety).

In another aspect, compounds comprising 1a, 1b, 2a, 2b, or 2c and polymer precursors may be produced through a series of synthetic steps. The embodiments are shown in Scheme 4 as products (11b1), (11b2), and (11b3) resulting from (10b):

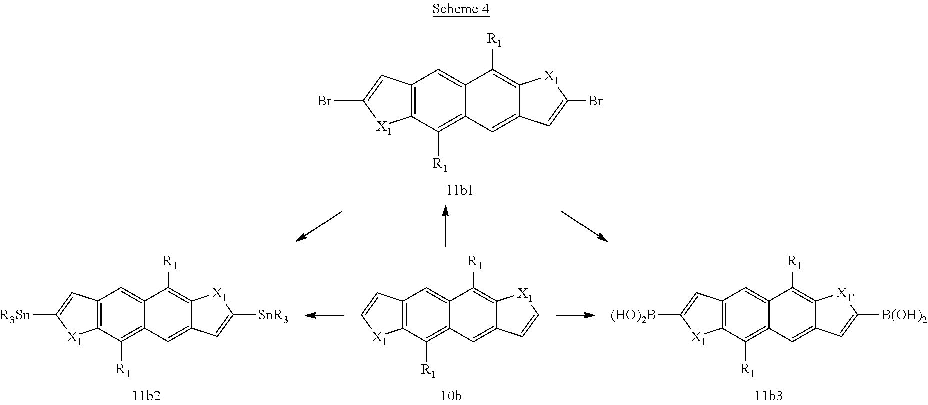

Wherein R₁ and X₁ are the same as described in Scheme 1 and R₃ is alkyl (X₂ is shown as —CH, but could be generalized to any X₂). Similarly, polymer precursors (11a1), (11a2), and (11a3) may be obtained from (10a), (10c), and (10d) with the appropriate chemical structure using the same synthetic procedures described below.

Possible routes from (10b) to (11b1) include the combination of (10b) (1.5 mmol) with NBS (3.6 mmol) in organic solvent (e.g., chloroform), stirring at room temperature for 24 hours, and subsequent washing (saturated sodium carbonate/water), extraction (DCM), drying with Na₂SO₄, and purification (Huang et al., 13 ORG. LETT. 5252 (2011), incorporated by reference in its entirety) or combination of (10b) with slow addition of PyHBr₃ (1 eq.) in solvent (THF/CHCl₃) and stirring for approx. 30 minutes at 0° C. The reaction is then diluted with dichloromethane and washed (2×100 mL Na₂S₂O₃), washed with brine, dried over Na₂SO₄, and purified by flash chromatography (gradient eluent 5% EtOAc/hexanes to 20% EtOAc/hexanes) (Qi et al., 133 J. AM. CHEM. SOC. 10050 (2011), incorporated by reference in its entirety, and Luo et al., 5 ORG. LETT. 4709-12 (2003), incorporated by reference in its entirety).

One possible route from (10b) to (11b2) is the combination of (10b) (5.52 mmol) with n-butyllithium (13.84 mmol, 2.5 M in hexane, added dropwise) under inert atmosphere in dry solvent (e.g., 12 mL hexane) at low temperatures (−78° C.), then allow to warm to room temperature, and subsequently cooled to −78° C. after about 20 minutes. A solution of tributylstannyl chloride (16.62 mmol) may be added dropwise, and then the solution can be brought to room temperature and stirred overnight. The mixture is then washed and the product washed, dried, and purified via column chromatography (dichloromethane:hexane=1:20 (v:v) (containing small amount triethylamine)) (Peng et al., 23 ADV. MATER. 4554 (2011), incorporated by reference in its entirety). A second route from (10b) to (11b2) is the combination of (10b) (1 eq.) with n-butyllithium (dropwise addition, 2.5 eq.) in tetramethylpiperidine (2.3 eq.) and THF (20 mL) at −78° C., then addition of R₃SnCl (3 eq.) at −78° C. The mixture may then be quenched with NaHCO₃, extracted with EtOAc, dried and purified by flash chromatography (Fargeas et al., 9 EUR. J. ORG. CHEM. 1711-21 (2003), incorporated by reference in its entirety).

A first route from (11b1) to (11b2) comprises combining (11b1) (0.80 mmol) with n-butyllithium (2 mmol) under inert atmosphere (argon) in dry solvent (THF) at low temperatures (−78° C.), then allow to warm to room temperature, and subsequently cooled to −78° C. after about 20 minutes. A solution of trimethylstannyl chloride (1.2 mmol 1.2 mL hexane) may be added dropwise, and then the solution can be brought to room temperature and stirred overnight. The mixture is then quenched with water, extracted with ether, dried, evacuated in vacuo, and recrystallized from isopropanol and hexanes (Peng et al., 23 ADV. MATER. 4554 (2011), incorporated by reference in its entirety). A second route from (11b1) to (11b2) comprises combining (11b1) with n-butyllithium (2.98 mmol) in 1-tert-butyl-6-methyl-2-bromo-3-cyclohexyl-1H-indole-1,6-dicarboxylate (2.29 mmol) and THF (35 mL) at −78° C., then addition of R₃SnCl (3.43 mmol) at −78° C., then allowing the mixture to warm to room temperature. The mixture may then be quenched with H₂O/NaHCO₃, extracted, dried and purified via flash chromatography (2:98 EtOAc/petroleum ether) (Avolio et al., 48 J. MED. CHEM. 4547 (2005), incorporated by reference in its entirety).

One possible route from (10b) to (11b3) is the combination of (10b) (3.24 mmol) with n-butyllithium (added dropwise, 2.88 mmol) in THF (35 mL) under inert atmosphere at room temperature and then stirred at ˜50° C. for 2 hours. Then 2-isopropoxy-4,4,5,5-tetramethyl-1,3,2-dioxaborolane (8.1 mmol) may be added in one portion at room temperature. After ˜6 hours, the reaction is stopped and the organic layer extracted (diethyl ether) and purified by column chromatography (Huo et al., 49 ANGEW CHEM. INT. ED. 1500 (2010), incorporated by reference in its entirety). A second route from (10b) to (11b3) is the combination of (1,5-cyclooctadiene)(methoxy)iridium(I) dimmer (0.15 eq.), 4,4′-di-tert-butyl-2,2′-dipyridyl (0.03 eq.), bis(pinacolato)diboron (2.00 eq.), 10b (1 eq.), and a stirring bar in a dry flask under argon. To this mixture is added anhydrous dichloromethane (2.2. mL) to give a colorless suspension and the flask is heated at 65° C. After ˜3 hours, the mixture is cooled to 23° C. and volatile removed under reduced pressure, and then the product is purified by flash chromatography (Schneider et al., 13 ORG. LETT. 3588 (2011) and Kolundzic et al., 133 J. AM. CHEM. SOC. 9104-11 (2011), both incorporated by reference).

Conversion from (11b1) to (11b3) may be accomplished by a number of routes. The first involves dissolving (11b1) (1.2 mmol) in anhydrous THF (25 mL) or an equivalent solvent and cooling to −78° C., then adding n-butyllithium (2.2. eq.) and stirring. Next, 2-isopropoxy-4,4,5,5-tetramethyl-1,3,2-dioxaborolane (4 eq.) may be added and the reaction stirred overnight. The product is extracted, washed, filtered and the solvent evaporated to give an oil that may be purified via column chromatography (e.g., 12″ silica column run with cyclohexane/ethyl acetate (4:1)) (Brookins et al. 19 J. MATER. CHEM. 4197 (2009), incorporated by reference in its entirety). Alternatively, to a solution of (11b1) (5.0 mmol in 400 mL 1,4-dioxane) may be added pinacolborane (357 mmol), then triethylamine 476 (mmol) dropwise at room temperature. After stirring for ˜3 hours, addition of (2-biphenyl)dicyclohexylphosphine (14.3 mmol) and Pd(OAc)₂ (3.57 mmol) is done. The combined mixture is then heated to about 85° C. for about 1.5 hours. Subsequently, a saturated aqueous NH₄Cl solution is added to the mixture and the product extracted with EtOAc, washed, dried and filtered, and then triturated in hexane:EtOAc (20:1) (Ikegashira et al. 49 J. MED. CHEM. 6950 (2006), incorporated by reference in its entirety).

In another aspect, compounds comprising 1a′, 1b′, 2a′, 2b′, or 2c′ may be produced through a series of synthetic steps. Compounds may be synthesized by synthetic routes that include processes analogous to those well-known in the chemical arts, particularly in light of the description contained herein. Scheme 5 exemplifies one way to form embodied compounds:

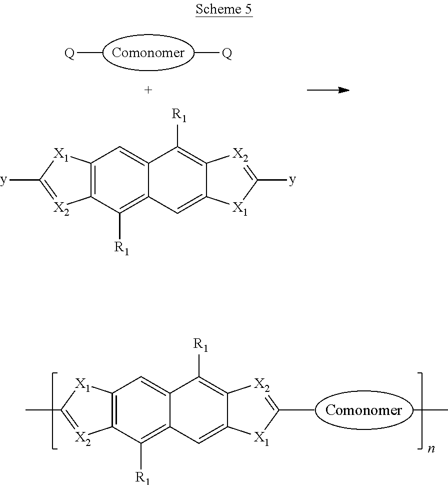

wherein n, X₁, X₂, y, R₁, n and comonomer all have the same meanings as above, k=m=1, and each Q is independently H, halo, OSO-alkyl, Mg-halo, Zn-halo, Sn(alkyl)₃, B(OH)₂, B(alkoxy)₂, OTs, or OTf. Similarly, polymers comprising 1b, 2a, 2b, or 2c may be made using the same synthetic procedures described herein. If the naphthalene reactant is (10a)-(10d), then (10a)-(10d) may be combined with dibromide comonomer (1:1 ratio), trans-di(μ-acetato)bis[o-(di-o-tolyl-phosphino)benzyl]dipalladium(II) (4% mol) and Cs₂CO₃ (2 eq.) and placed in a microwave vial with a magnetic stirring bar. The vial is then sealed with a cap and purged with nitrogen to remove the oxygen. THF is added and the reaction is heated with an oil bath at 120° C. (reaction under pressure). At the end of the reaction time, the reaction is cooled and the corresponding 5-alkyl[3,4-c]pyrrole-4,6-dione is added in excess as a capping agent. The solution was heated again at 120° C. for 1 hour to complete the end-capping procedure. After cooling, the mixture is poured in to 500 mL of cold methanol to precipitate to product. The precipitate is filtered, extracted via Soxhlet extraction with acetone followed by hexanes to remove catalytic residue and low MW materials. Polymers may be extracted with chloroform and then re-precipitated by re-pouring into cold methanol and filtering (Berrouard et al., 50 ANGEW. CHEM. INT. ED. 1-5 (2011), incorporated by reference in its entirety).

Alternatively, if the naphthalene reactant is (11b1) or (11a1), the polymer may be formed by combining (11b2) (or (11a2)) (0.25 mmol) with ditin, or diboranes or diboronate esters (comonomer) (1:1 eq.) in toluene (15 mL). The solution is flushed with argon for 10 min, and then Pd₂DBA₃ (2 mol %) and P(o-tolyl)₃ (16.36 mg, 8%) are added into the flask. The flask is purged, heated to 110° C., and stirred for 48 h under argon. 2-Tributylstannyl thiophene (20 μL) is then added to the reaction and after two hours, 2-bromothiophene (6.3 μL) is added and the mixture is stirred overnight to complete the end-capping reaction. The mixture is then cooled to room temperature, and the product filtered, washed with methanol (350 mL) and hexane in a Soxhlet apparatus to remove the oligomers and catalyst residue. Finally, the polymer is extracted with chloroform, condensed by evaporation and precipitated into methanol. The polymer was collected as a dark purple solid (Peng et al., 23 ADV. MATER. 4554 (2011), incorporated by reference in its entirety and Huo et al., 49 ANGEW CHEM. INT. ED. 1500 (2010), incorporated by reference in its entirety).

A second alternative for Scheme 5 is to start with (11b2) or (11a2). For example, (11b2) (0.25 mmol) and the dibromide comonomer (1:1 eq.) are dissolved in toluene (15 mL). The solution is flushed with argon for 10 min, and then Pd₂DBA₃ (2 mol %) and P(o-tolyl)₃ (16.36 mg, 8%) are added into the flask. The flask is purged, heated to 110° C., and stirred for 48 h under argon. 2-Tributylstannyl thiophene (20 μL) is then added to the reaction and after two hours, 2-bromothiophene (6.3 μL) is added and the mixture is stirred overnight to complete the end-capping reaction. The mixture is then cooled to room temperature, and the product filtered, washed with methanol (350 mL) and hexane in a Soxhlet apparatus to remove the oligomers and catalyst residue. Finally, the polymer is extracted with chloroform, condensed by evaporation and precipitated into methanol. The polymer was collected as a dark purple solid (Peng et al., 23 ADV. MATER. 4554 (2011), incorporated by reference in its entirety).

A third alternative for Scheme 3 is to start with (11b3) or (11a3). For example, (11b3) (0.35 mmol) and dibromide comonomer (1:1 eq.) are dissolved in toluene (15 mL) with sodium carbonate (1M, 3 mL). The solution is flushed with argon for 10 min, and then Pd(PPh₃)₄ (15 mg) is added into the flask. The flask is purged, heated to 110° C., and stirred for 18 h under argon. The mixture is then cooled to room temperature, and the product filtered, washed with methanol (100 mL) and hexane in a Soxhlet apparatus to remove the oligomers and catalyst residue. Finally, the polymer is extracted with chloroform, condensed by evaporation and precipitated into methanol. The polymer was collected as a dark purple solid (Huo et al., 49 ANGEW CHEM. INT. ED. 1500 (2010), incorporated by reference in its entirety).

Scheme 6 exemplifies another possible way to form embodied compounds:

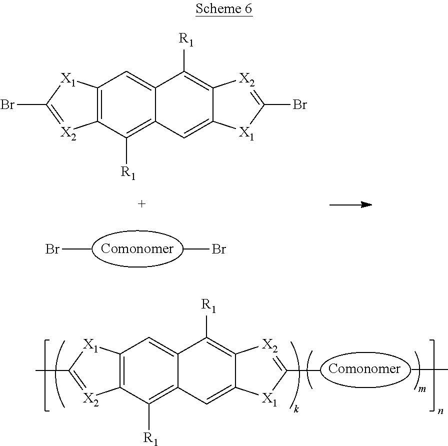

wherein n, X₁, X₂, y, R₁, n and comonomer all have the same meanings as above, and k>1 and m>1.

Starting with (11b1) or (11a1), the polymer in Scheme 6 may be formed by combining (11ba1) (or (11a1), dibromide comonomer (1:1 eq.), zinc powder (3.1 eq.), triphenyl phosphine (1 eq.), bipyridine (0.075 eq.), and nickel (II) chloride (0.075 eq.) in a dry round bottom flask inside of a dry box. The flask is sealed with a septum and removed from the dry box and anhydrous DMAC is added via cannulation. The reaction is heated to 85° C. and after about five minutes, the reaction has a green-yellow color, with the yellow growing in intensity overtime. The reaction may be run for 24 hours, and then the polymer is precipitated into methanol and dried under vacuum.

Scheme 7 exemplifies another possible way to form embodied compounds:

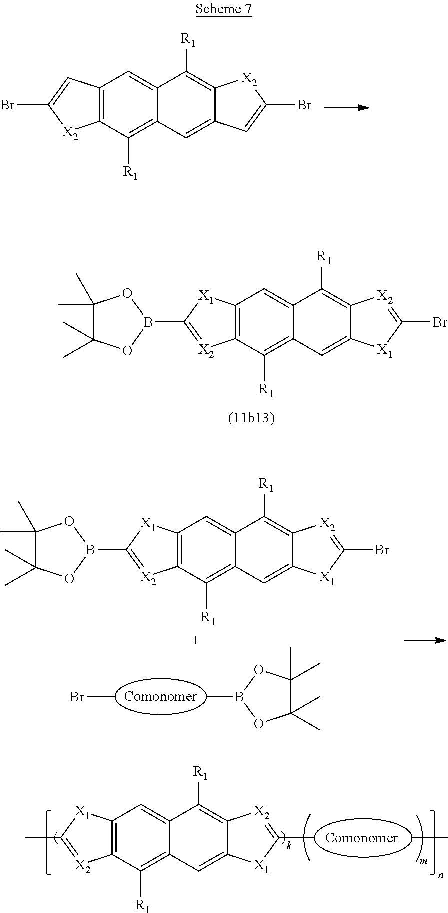

wherein n, X₁, X₂, y, R₁, n and comonomer all have the same meanings as above, and k>1 and m>1.

Starting with (11b1) or (11a1), the polymer in Scheme 5 may be formed by first dissolving (11b1) (or (11a1)) in anhydrous THF and then adding n-butyllithium (1.2 eq.) and stirring. 2-Isopropoxy-4,4,5,5-tetramethyl-1,3,2-dioxaborolane (4 eq.) is added and the reaction stirred overnight. The product was extracted, washed, dried and evaporated give (11b13), which can be purified via with cyclohexane/ethyl acetate (4:1) column chromatography. Compound (11b13) is combined with cesium fluoride (7 eq.) in a dry flask purged with argon and then the comonomer is added in degassed solvent (12 mL per mmol of monomer) along with Pd₂dba₃ (2 mol %) and tri(t-butyl)phosphonium tetrafluoroborate (6 mol %), and the reaction is refluxed for 48 h. The polymer is precipitated into methanol and dried under vacuum.

Comonomers may be produced by known synthetic methods. Such methods are shown in, for example, 72 J. ORG. CHEM. 442-451 (2007), 6 BEILSTEIN J. ORG. CHEM. 830-845 (2010), Jerry March, Michael B. Smith, MARCH'S ADVANCED ORGANIC CHEMISTRY: REACTIONS, MECHANISMS, AND STRUCTURE (6^{th} Ed. Wiley-Interscience), Richard C. Larock, COMPREHENSIVE ORGANIC TRANSFORMATIONS (1999 Wiley-VCH), all hereby incorporated by reference in their entireties.

In another aspect, embodiments herein are optimized for reorganization energy and mobility. In some embodiments, compounds embodied herein have improved solid state properties as a result of lower reorganization energy and/or higher mobility. In some embodiments, the properties of the compounds embodied herein may be described by Marcus theory (R. A. Marcus, 65 REV. MOD. PHYS. 599 (1993), herein incorporated by reference in its entirety).

Charge transport properties depend critically on the degree of ordering of the system or molecular ordering in the solid state, as well as the density of chemical impurities and/or structural defects such as grain size and dislocations. At the electronic level, two of the most important factors that control transport properties in organic conjugated materials are the interchain transfer integral β, and the reorganization energy λ. The transfer integral expresses the ease of transfer of a charge between interacting chains. The reorganization energy term describes the strength of the electron-phonon coupling. It is proportional to the geometric relaxation energy of the charged molecule over the individual neutral unit. In the context of semi-classical electron-transfer theory, the electron-transfer (hopping) rate can be expressed from Marcus theory in a simplified way as:

$${k}_{et} = \frac{4 {π}^{2}}{h} \frac{1}{\sqrt{4 π {k}_{B} λ T}} {β}^{2} {e}^{- \frac{λ}{4 {k}_{B} T}} \qquad ( 1 )$$

(R. A. Marcus, 65 REV. MOD. PHYS. 599 (1993), herein incorporated by reference in its entirety) where T is the temperature, λ is the reorganization energy, β is the transfer integral, and h and k_{B} are the Planck and Boltzmann constants, respectively.

It is possible to simplify equation (1) to:

$${k}_{et}^{simple} = \frac{1}{\sqrt{λ}} {β}^{2} {e}^{- λ} \qquad ( 2 )$$

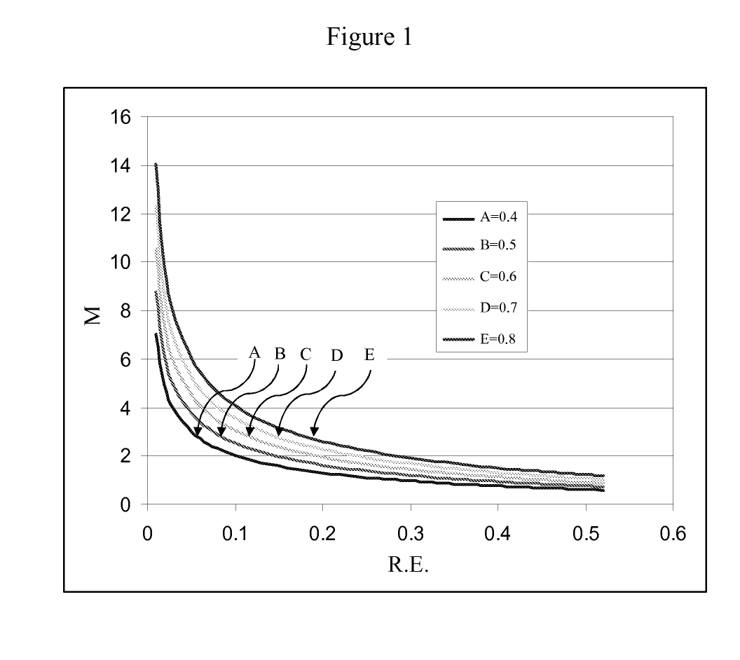

In order to characterize the relative influence of both parameters λ and β to the charge transport rate. FIG. 1 schematically depicts the relationship of mobility (M) as a function of the reorganization energy (R.E.) at five different values of the transfer integral (ranging from 0.4 eV to 2 eV). From FIG. 1, it is clear that the difference in mobility for different transfer integrals is only significant for small values of the reorganization energy. A big increase in the transfer integral does not yield a significant variation in the mobility, unless the reorganization energies are small. This implies that any optimization of the mobility should start with the design of single molecules with very low reorganization energy.

The reorganization energy includes two contributions that are associated with charge hopping. One is introduced by the geometric changes within the single molecule, and is denoted the internal part. The second one arises from the repolarization changes of the surrounding medium and is usually much smaller than the first one. In studies to qualitatively order molecules it is generally valid to neglect this last contribution in the evaluation of the reorganization energy as no significant solvent reorganization occurs during the charge transfer in the condensed phase.

Table 1 incorporates reorganization energies for a number of embodiments. For each molecule, the geometry is optimized using quantum mechanics for both neutral and ionic states. Consequently, the basic hopping step in a molecular wire is defined by four energies: E₀ and E₊ represent the energies of the neutral and cation species in their lowest energy geometries, respectively, while E₀* and E₊* represent the energies of the neutral and cation species with the geometries of the cation and neutral species, respectively.

The quantum mechanics calculations to determine these above mentioned quantities used the experimentally parameterized Hamiltonian PM6 implemented in VAMP® semi-empirical molecular orbital software (Accelrys Software Inc.). Pentacene was used as the reference to validate the Hole Reorganization Energy calculations. Experimental data for Pentacene RE was ˜0.12 eV (see M. Malagoli and J. L. Bredas, 327 CHEM. PHYS. LETT. 13 (2000) and N. W. Gruhn et al., 89 PHYS. REV. LETT. 275503 (2002), both hereby incorporated by reference in their entirety), compared to 0.114 eV from our calculations based on VAMP® (Accelrys Software Inc.).

Hole Reorganization energies for embodiments may comprise from about 0 eV to about 0.5 eV. In some embodiments, the hole reorganization energy is from about 0.04 to about 0.35 eV. In some embodiments, the hole reorganization energy is 0.35 eV or less. In some embodiments, the hole reorganization energy is about 0.04, 0.05, 0.06, 0.07, 0.08, 0.09, 0.10, 0.11, 0.12, 0.13, 0.14, 0.15, 0.17, 0.19, 0.20, 0.22, 0.25, 0.27, 0.30, 0.31, 0.32, 0.33, 0.34, 0.35, 0.37, 0.40, 0.45, or 0.50.

| | Vertical | Vertical | |
|---|---|---|---|
| | Ionization | Electron | Hole |
| | Potential | Affinity | Reorganization |
| Compound | [eV] | [eV] | Energy [eV] |
| 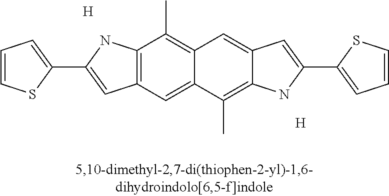 | 6.78 | −1.62 | 0.0766 |
| 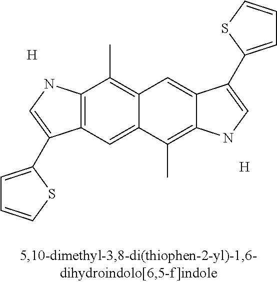 | 6.82 | −.145 | 0.0792 |
| 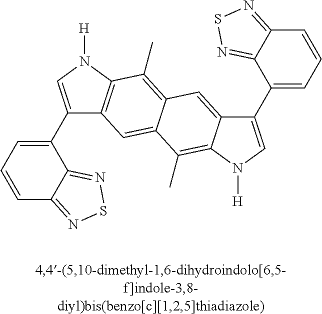 [SMILES: Cc1c2cc3c(-c4cccc5nsnc45)c[nH]c3c(C)c2cc2c(-c3cccc4nsnc34)c[nH]c12] | 6.75 | −2.14 | 0.113 |
| 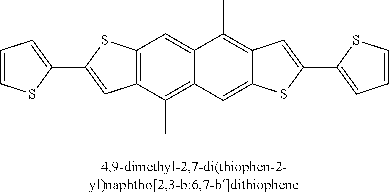 [SMILES: Cc1c2cc(-c3cccs3)sc2cc2c(C)c3cc(-c4cccs4)sc3cc12] | 7.45 | −2.01 | 0.112 |
| 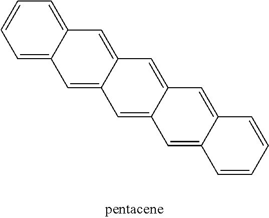 [SMILES: c1ccc2cc3cc4cc5ccccc5cc4cc3cc2c1] | 7.75 | −1.67 | 0.114 |
| 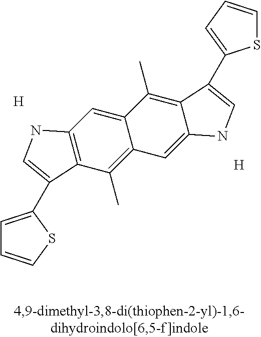 | 6.80 | −1.38 | 0.117 |
| 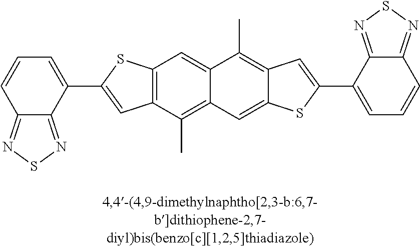 [SMILES: Cc1c2cc(-c3cccc4nsnc34)sc2cc2c(C)c3cc(-c4cccc5nsnc45)sc3cc12] | 7.53 | −2.55 | 0.119 |
| 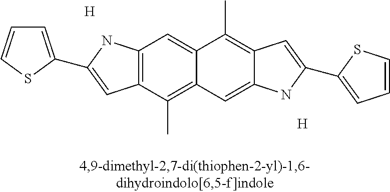 | 6.80 | −1.61 | 0.134 |
| 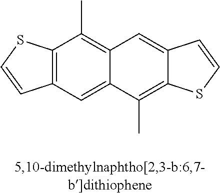 [SMILES: Cc1c2cc3ccsc3c(C)c2cc2ccsc12] | 7.45 | −1.59 | 0.139 |
| 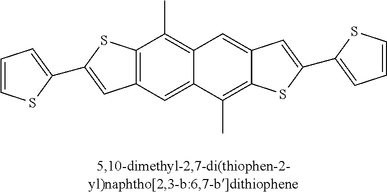 [SMILES: Cc1c2cc3cc(-c4cccs4)sc3c(C)c2cc2cc(-c3cccs3)sc12] | 7.39 | −2.01 | 0.142 |
| 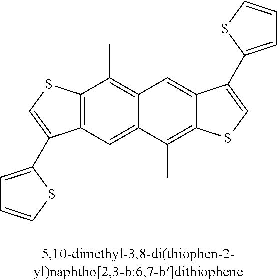 [SMILES: Cc1c2cc3c(-c4cccs4)csc3c(C)c2cc2c(-c3cccs3)csc12] | 7.35 | −1.74 | 0.143 |
| 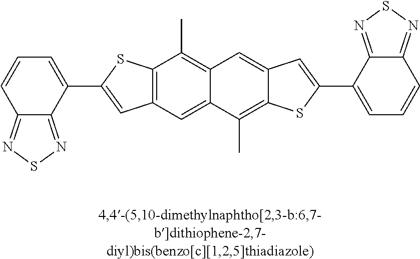 [SMILES: Cc1c2cc3cc(-c4cccc5nsnc45)sc3c(C)c2cc2cc(-c3cccc4nsnc34)sc12] | 7.52 | −2.55 | 0.158 |
| 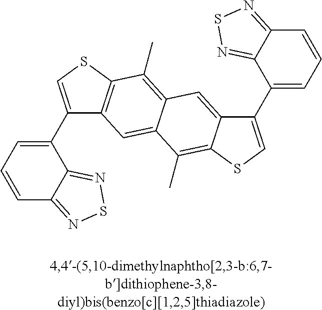 [SMILES: Cc1c2cc3c(-c4cccc5nsnc45)csc3c(C)c2cc2c(-c3cccc4nsnc34)csc12] | 7.29 | −2.32 | 0.159 |
| 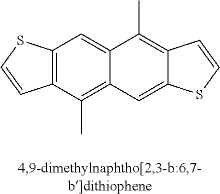 [SMILES: Cc1c2ccsc2cc2c(C)c3ccsc3cc12] | 7.47 | −1.60 | 0.164 |
| 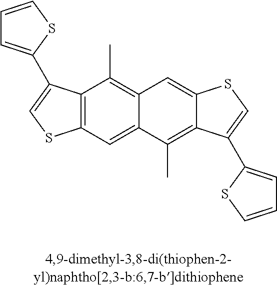 [SMILES: Cc1c2cc3scc(-c4cccs4)c3c(C)c2cc2scc(-c3cccs3)c12] | 7.36 | −1.76 | 0.168 |
| 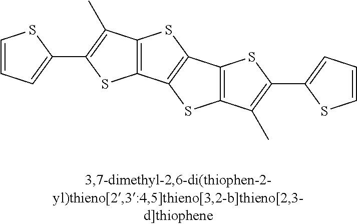 [SMILES: Cc1c(-c2cccs2)sc2c1sc1c3sc(-c4cccs4)c(C)c3sc21] | 7.35 | −2.55 | 0.206 |
| 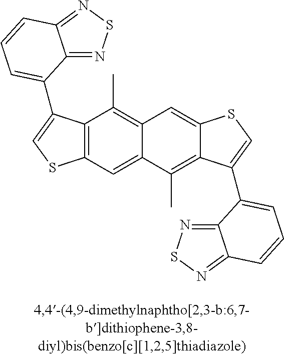 [SMILES: Cc1c2cc3scc(-c4cccc5nsnc45)c3c(C)c2cc2scc(-c3cccc4nsnc34)c12] | 7.33 | −2.33 | 0.214 |
| 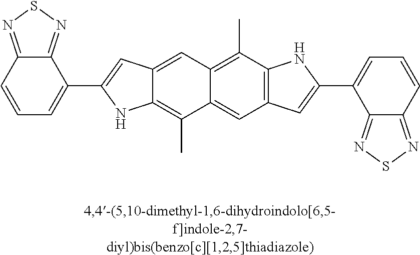 [SMILES: Cc1c2cc3cc(-c4cccc5nsnc45)[nH]c3c(C)c2cc2cc(-c3cccc4nsnc34)[nH]c12] | 6.93 | −2.44 | 0.228 |
| 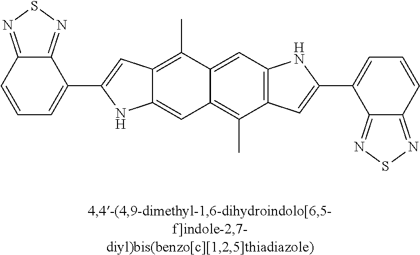 [SMILES: Cc1c2cc(-c3cccc4nsnc34)[nH]c2cc2c(C)c3cc(-c4cccc5nsnc45)[nH]c3cc12] | 6.94 | −2.44 | 0.27 |
| 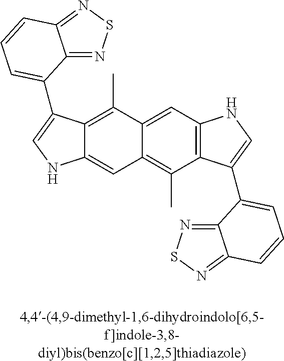 [SMILES: Cc1c2cc3[nH]cc(-c4cccc5nsnc45)c3c(C)c2cc2[nH]cc(-c3cccc4nsnc34)c12] | 6.85 | −1.95 | 0.28 |
| 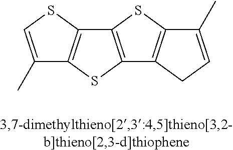 | 7.63 | −2.06 | 0.307 |

The compositions described herein (monomers, oligomers, polymers) can be used to make a wide variety of devices. For example, the device can be a fused thiophene moiety-containing composition configured in an electronic, optoelectronic, or nonlinear optical device. The compositions described herein can also be used in field effect transistors (FETs), thin-film transistors (TFTs), organic light-emitting diodes (OLEDs), PLED applications, electro-optic (EO) applications, as conductive materials, as two photon mixing materials, as organic semiconductors, as non-linear optical (NLO) materials, as RFID tags, as electroluminescent devices in flat panel displays, in photovoltaic devices, and as chemical or biological sensors.

The polymers comprising the fused thiophene moieties described herein (1a′, 1b′, 2a′, 2b′, 2c′, and 2d′) possess several advantages over similar compounds. The polymers embodied herein are easier to modify on the designed fused rings, allowing for improvements in the polymerization process and processibility. Further, substituents can be introduced to multiple positions which can enable fine tuning material packing behaviors. The introduction of substituted pyrrole structures into substituted naphthalene results in lower reorganization energy and higher mobility for the compounds and finally β-substituents on the five-member ring increases the material stability of the resulting polymers.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Although specific starting materials and reagents are depicted in the Examples below, other starting materials and reagents can be easily substituted to provide a variety of derivatives and/or reaction conditions. In addition, many of the compounds prepared by the methods described below can be further modified in light of this disclosure using conventional chemistry well known to those skilled in the art. Only reasonable and routine experimentation will be required to optimize such process conditions.

### Example 1

### 1,5-dibromonaphthalene-2,6-diol

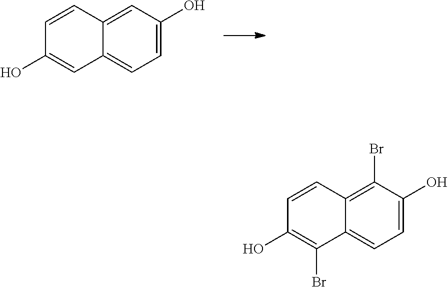 [SMILES: Oc1ccc2cc(O)ccc2c1 ; Oc1ccc2c(Br)c(O)ccc2c1Br]

To a solution of naphthalene-2,6-diol (1) (5.1 g) in 50 mL of Tetrahydrofuran (THF), was added N-Bromosuccinimide (NBS, 11.4 g). The mixture was refluxing and monitored by GCMS. The reaction was quenched with saturated sodium thiosulfate, and filtered. The solid was washed by water to afford 1,5-dibromonaphthalene-2,6-diol (90%). LRMS (ESI): Calcd. for C₁₀H₆Br₂O₂: 317.8714. Found: 317.9.

### Example 2

### 1,5-Dibromo-2,6-bis(methoxymethoxy)naphthalene

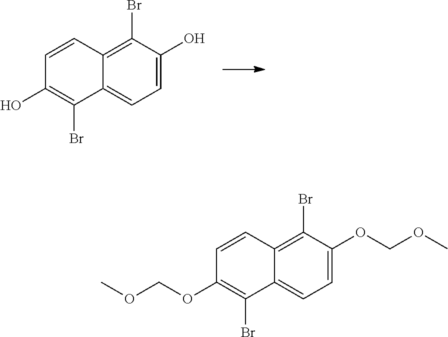

To a solution of 1,5-dibromonaphthalene-2,6-diol (2) (77.15 g) in dichloromethane (500 mL), diisoprorylethylamine (255 mL) and chloro(methoxy)methane (MOMCl, 98.6 g) were added at 0° C. After stirring for 22 h at room temperature, the reaction was quenched by adding water. The crude products were extracted with ethyl acetate and the combined organic extracts were washed with brine, dried over sodium sulfate (Na₂SO₄), and concentrated in vacuum. The solid residue was stirred in n-hexanes to afford analytically pure 1,5-dibromo-2,6-bis(methoxymethoxy)naphthalene (90%). LRMS (ESI): Calcd. for C₁₄H₁₄Br₂O₄: 405.9238. Found: 406.0.

### Example 3

### 2,6-bis(methoxymethoxy)-1,5-dimethylnaphthalene

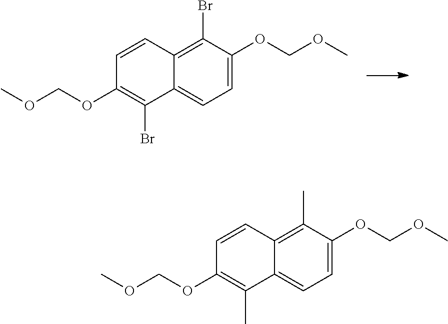

1,5-dibromo-2,6-bis(methoxymethoxy)naphthalene (100 g) was dissolved in THF (1.6 L) and treated with n-Butyllithium (n-BuLi, 246 mL of 2.5 M solution in hexane, 2.5 e.g.) at −78° C. and stirred for one hour. The resulting mixture was quenched with iodomethane (46 mL) for 0.5 hour. The solution was extracted with ethyl acetate and Na₂S₂O₃ and NaHCO₃. The mixture was evaporated under reduced vacuum to give the product 1,5-dihexyl-2,6-bis(methoxymethoxy)naphthalene (90%). LRMS (ESI): Calcd. for C₁₆H₂₀O₄: 276.1362. Found: 276.1.

### Example 4

### 3,7-dibromo-2,6-bis(methoxymethoxy)-1,5-dimethylnaphthalene

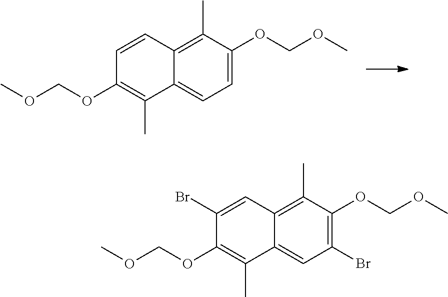

N-BuLi (140 mL of 2.5 M solution) was added at room temperature to a solution of 2,6-bis(methoxymethoxy)-1,5-dimethylnaphthalene (29 g) in anhydrous ethyl ether (Et₂O, 1 L). After 3 h, the solution was cooled to 0° C. A THF (100 ml) solution of 1,2-dibromo-1,1,2,2-tetrafluoroethane (108 g) was added to the above mixture, and the resulting mixture was allowed to warm to room temperature over 4 h. Saturated sodium thiosulfate was then added to quench the reaction, and the aqueous layer was extracted with ethyl acetate. The combined organic extracts were washed with brine and dried over Na₂SO₄. After evaporation, the resulting crude product was purified by column chromatography on silica gel to give the final product 3,7-dibromo-2,6-bis(methoxymethoxy)-1,5-dimethylnaphthalene (45%). LRMS (ESI): Calcd. for C₁₆H₁₈Br₂O₄: 433.9551. Found: 434.0.

### Example 5

### 3,7-dibromo-1,5-dimethylnaphthalene-2,6-diol

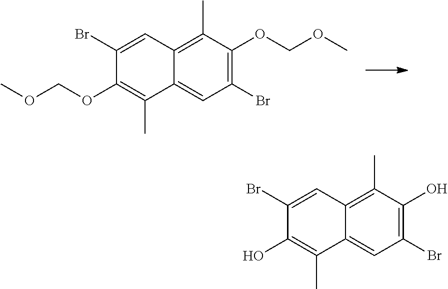

A mixture of 3,7-dibromo-2,6-bis(methoxymethoxy)-1,5-dimethyl naphthalene (3 g) and hydrochloric acid (6N HCl, 30 mL) in dichloromethane/methanol (15 ml/75 ml) was heated to 50° C. and stirred overnight. The resulting mixture was poured into water and extracted with ethyl acetate. The organic extracts were washed with brine and dried over Na₂SO₄. The solvents were evaporated to give the crude product 3,7-dibromo-1,5-dimethylnaphthalene-2,6-diol. (90%). LRMS (ESI): Calcd. for C₁₂H₁₀Br₂O₂: 345.9027. Found: 345.9.

### Example 6

### 3,7-Dibromo-1,5-dimethylnaphthalene-2,6-diyl bis(trifluoro methanesulfonate)

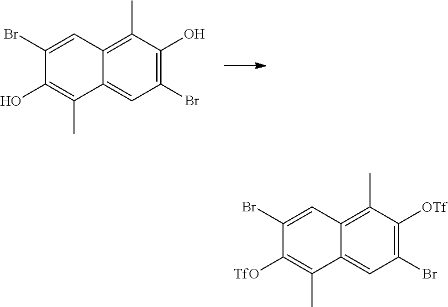 [SMILES: Cc1c(O)c(Br)cc2c(C)c(O)c(Br)cc12 ; Cc1c(OS(=O)(=O)C(F)(F)F)c(Br)cc2c(C)c(OS(=O)(=O)C(F)(F)F)c(Br)cc12]

To a suspension of 3,7-dibromo-1,5-dimethylnaphthalene-2,6-diol (3.24 g), pyridine (4.5 mL) in dichloromethane (90 mL) was slowly added trifluoromethanesulfonic anhydride (3.6 mL) at 0° C. After the mixture was stirred for 4 h at room temperature, water and hydrochloric acid (1 M) were added. The resulting mixture was extracted with dichloromethane and combined organic layer was dried (MgSO₄) and concentrated in vacuo. The residue was purified by column chromatography on silica gel to give 3,7-Dibromo-1,5-dimethylnaphthalene-2,6-diyl bis(trifluoromethanesulfonate) (80%). LRMS (ESI): Calcd. for C₁₄H₈Br₂F₆O₆S₂: 609.8013. Found: 609.9.

### Example 7

### ((3,7-dibromo-1,5-dimethylnaphthalene-2,6-diyl)bis(ethyne-2,1-diyl))bis(triisopropylsilane)

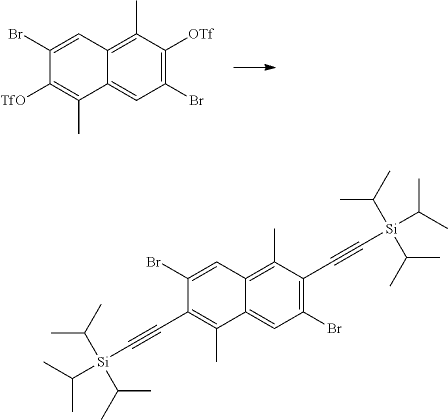 [SMILES: Cc1c(OS(=O)(=O)C(F)(F)F)c(Br)cc2c(C)c(OS(=O)(=O)C(F)(F)F)c(Br)cc12 ; Cc1c(C#C[Si](C(C)C)(C(C)C)C(C)C)c(Br)cc2c(C)c(C#C[Si](C(C)C)(C(C)C)C(C)C)c(Br)cc12]

To a degassed solution of 3,7-Dibromo-1,5-dimethylnaphthalene-2,6-diyl bis(trifluoro methanesulfonate) (28 mg) in dimethylformamide (DMF, 1 mL) and diisopropylamine (0.7 mL) was added bis(triphenylphosphine) palladium chloride (Pd(PPh₃)₂Cl₂, 25 mg), copper(I) iodide (CuI) (9 mg) and ethynyltriisopropylsilane (70 μL). After the mixture was stirred for 50 min at room temperature, water and hydrochloric acid (1 M) were added. The resulting mixture was extracted with ethyl acetate and the combined organic layer was dried (MgSO₄) and concentrated in vacuo. The residue was purified by column chromatography on silica gel to give ((3,7-dibromo-1,5-dimethylnaphthalene-2,6-diyl)bis(ethyne-2,1-diyl))bis(triisopropylsilane) (50%). LRMS (ESI): Calcd. for C₃₄H_{SO}Br₂Si₂: 631.1250 (MW-pr). Found: 631.1 (MW-43).

### Example 8

### 4,8-dimethyl-N₂,N₆-dipentyl-3,7-bis((triisopropylsilyl) ethynyl) naphthalene-2,6-diamine

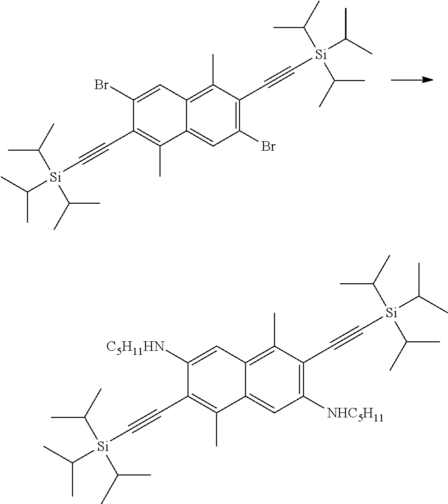

A solution of ((3,7-dibromo-1,5-dimethylnaphthalene-2,6-diyl)bis(ethyne-2,1-diyl))bis(triisopropylsilane) (0.73 mmol), t-NaOBu (304 mg), Pd₂dba₃ (54 mg) and (S)-BINAP (78 mg) in dry toluene (8 mL) was purged with nitrogen for 10 min. Pentan-1-amine (0.58 ml) was added via a syringe and the mixture was refluxed under nitrogen for 4 hours. After cooling to room temperature, water was added to the solution and the reaction mixture was extracted twice with diethyl ether. After the organic phases were dried over MgSO₄, the solvents were removed using a rotary evaporator. The residue was purified by column chromatography on silica gel to give 4,8-dimethyl-N2,N6-dipentyl-3,7-bis((triisopropylsilyl)ethynyl)naphthalene-2,6-diamine (40%). LRMS (ESI): Calcd. For C₄₄H₇₄N₂Si₂: 686.5391. Found: 686.6.

### Example 9

### 3,7-di(hex-1-yn-1-yl)-1,5-dimethylnaphthalene-2,6-diol

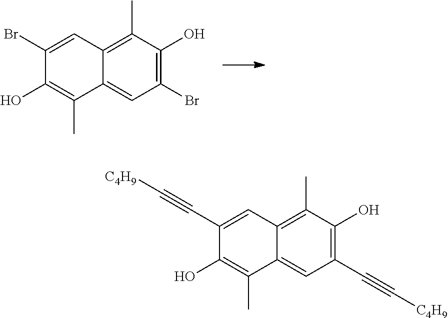

Compounds 3,7-dibromo-1,5-dimethylnaphthalene-2,6-diol (560 mg), [Pd(PPh₃)₄] (31.0 mg), CuI (24 mg), PPh₃ (31 mg), iPr₂NH (5.8 mL), and Et₃N (16.1 mL) were added to a three necked flask, equipped with a condenser and a magnetic stirrer under an inert atmosphere. The mixture was purged with Ar and stirred for 30 min, while compound hex-1-yne (1.1 mL) was added in one portion. After the addition, the reaction mixture was slowly heated to 80° C. and stirred for 15 min at this temperature. After cooling to room temperature, the solvent was removed under reduced pressure to afford the residue, which was extracted with DCM, and washed twice with water. The organic layer was dried (MgSO₄). After removal of solvent, the product was purified by flash column chromatography to give 3,7-di(hex-1-yn-1-yl)-1,5-dimethylnaphthalene-2,6-diol (90%). LRMS (ESI): Calcd. For C₂₄H₂₈O₂: 348.2089. Found: 348.2.

### Example 10

### 3,7-di(hex-1-yn-1-yl)-1,5-dimethylnaphthalene-2,6-diyl bis(trifluoromethanesulfonate)

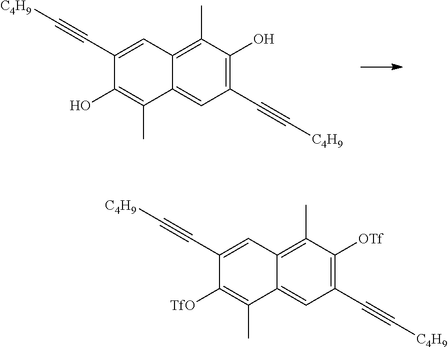

To a suspension of 3,7-di(hex-1-yn-1-yl)-1,5-dimethylnaphthalene-2,6-diol (210 mg), pyridine (0.3 mL) in dichloromethane (6 mL) was slowly added trifluoromethanesulfonic anhydride (0.21 mL) at 0° C. After the mixture was stirred for 2 h at room temperature, water and hydrochloric acid (1 M) were added. The resulting mixture was extracted with dichloromethane and combined organic layer was dried (MgSO₄) and concentrated in vacuo. The residue was purified by column chromatography to give 3,7-di(hex-1-yn-1-yl)-1,5-dimethylnaphthalene-2,6-diyl bis(trifluoromethanesulfonate) (40%) LRMS (ESI): Calcd. For C₂₆H₂₆F₆O₆S₂: 612.1075. Found: 612.0.

### Example 11

### 3,7-di(hex-1-yn-1-yl)-1,5-dimethyl-N2,N6-dipentylnaphthalene-2,6-diamine

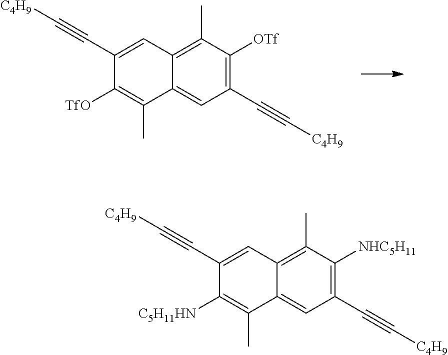

A solution of 3,7-di(hex-1-yn-1-yl)-1,5-dimethylnaphthalene-2,6-diylbis(trifluorometh-anesulfonate) (367 mg), Cs₂CO₃ (960 mg), Pd₂dba₃ (133 mg) and (S)-BINAP (385 mg) in dry toluene (7 ml) was purged with nitrogen for 20 min. Pentan-1-amine (0.48 mL) was added via a syringe and the mixture was refluxed under nitrogen for 7 h. After cooling to room temperature, water was added to the solution and the reaction mixture was extracted twice with diethyl ether. After the organic phases were dried over MgSO4, the solvents were removed using a rotary evaporator. The crude product was purified by column chromatography and the desired product of 3,7-di(hex-1-yn-1-yl)-1,5-dimethyl-N2,N6-dipentylnaphthalene-2,6-di-amine was obtained (24%). LRMS (ESI): Calcd. For C₃₄H₅₀N₂: 486.3974. Found: 486.4.

### Example 12

### 2,7-dibutyl-5,10-dimethyl-1,6-dipentyl-1,6-dihydroindolo[6,5-1]indole

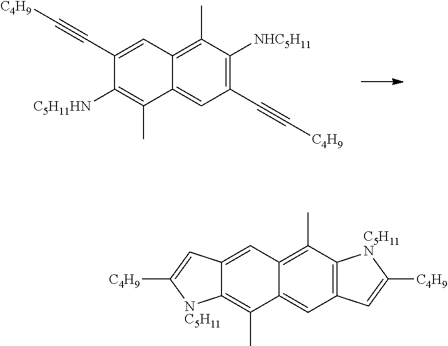

In an dried pressure tube, to a solution of 3,7-di(hex-1-yn-1-yl)-1,5-dimethyl-N₂,N₆-dipentylnaphthalene-2,6-diamine (10 mg) in DMSO (2 mL) was added finely crushed KOH (100 mg). The resulting reaction mixture was heated at 120° C. for 16 h and was extracted with ethyl acetate and the combined organic layer was dried (MgSO₄) and concentrated in vacuo. The residue was purified by column chromatography on silica gel to give 4,9-dimethyl-1,6-dipentyl-1,6-dihydroindolo[6,5-f]indole. LRMS (ESI): LRMS (ESI): Calcd. For C₃₄H₅₀N₂: 486.3974. Found: 486.4.

### Example 13

### 1,5-dibromonaphthalene-2,6-diylbis(trifluoromethanesulfonate)

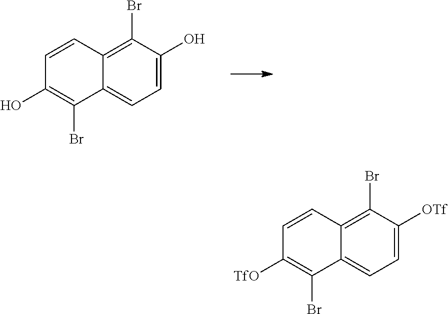 [SMILES: Oc1ccc2c(Br)c(O)ccc2c1Br ; O=S(=O)(Oc1ccc2c(Br)c(OS(=O)(=O)C(F)(F)F)ccc2c1Br)C(F)(F)F]

To a suspension of 1,5-dibromonaphthalene-2,6-diol (10.1 g), pyridine (15.3 mL) in dichloromethane (305 mL) was slowly added trifluoromethanesulfonic anhydride (12.2 mL) at 0° C. After the mixture was stirred for 8 h at room temperature, water and hydrochloric acid (1 M) were added. The resulting mixture was extracted with dichloromethane and combined organic layer was dried (MgSO₄) and concentrated in vacuo. The residue was purified by column chromatography to give 1,5-dibromonaphthalene-2,6-diyl bis(trifluoromethanesulfonate) (50%) LRMS (ESI): Calcd. For C₁₂H₄Br₂F₆O₆S₂: 581.7700. Found: 581.8.

### Example 14

### 1,5-dibromo-2,6-di(hex-1-yn-1-yl)naphthalene

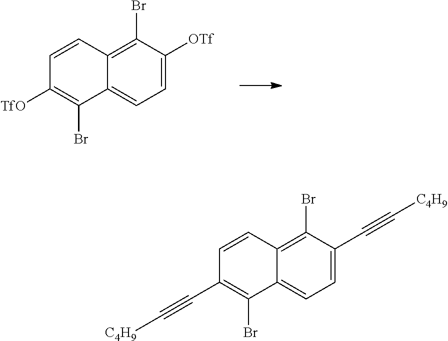

To a degassed solution of 1,5-dibromonaphthalene-2,6-diyl bis(trifluoromethanesulfonate) (582 mg) in DMF (7 mL) and diisopropylamine (7 mL) was added Pd(PPh₃)₂Cl₂ (70 mg), CuI (38 mg) and hex-1-yne (222 μL). Reaction was stirred at room temperature and monitored by GCMS. Water and hydrochloric acid (1 M) were added. The resulting mixture was extracted with dichloromethane and the combined organic layer was dried (MgSO₄) and concentrated in vacuo. The residue was purified by column chromatography on silica gel eluted with hexane to give 1,5-dibromo-2,6-di(hex-1-yn-1-yl)naphthalene (50%) LRMS (ESI): Calcd. For C₂₂H₂₂Br₂: 446.2181. Found: 446.1.

### Example 15

### 2,6-di(hex-1-yn-1-yl)-N1,N5-dipentylnaphthalene-1,5-diamine

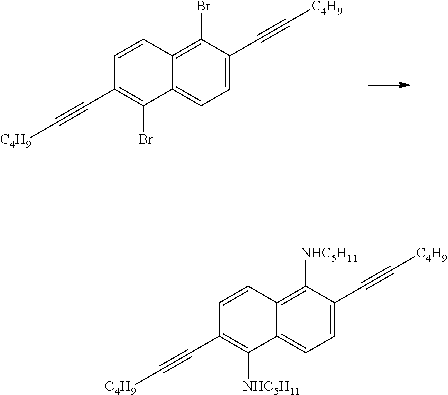

A solution of 1,5-dibromo-2,6-di(hex-1-yn-1-yl)naphthalene (710 mg), t-BuONa (367 mg), Pd₂dba₃ (73 mg) and (S)-BINAP (198 mg) in dry toluene (6 ml) was purged with nitrogen for 20 min. Pentan-1-amine (0.58 mL) was added via a syringe and the mixture was refluxed under nitrogen for 7 h. After cooling to room temperature, water was added to the solution and the reaction mixture was extracted twice with diethyl ether. After the organic phases were dried over MgSO₄, the solvents were removed using a rotary evaporator. The crude product was purified by column chromatography and the desired product of 2,6-di(hex-1-yn-1-yl)-N₁,N₅-dipentylnaphthalene-1,5-diamine was obtained (50%). LRMS (ESI): Calcd. For C₃₂H₄₆N₂: 458.3661. Found: 458.4.

### Example 16

### 2,7-dibutyl-3,8-dipentyl-3,8-dihydroindolo[7,6-g]indole

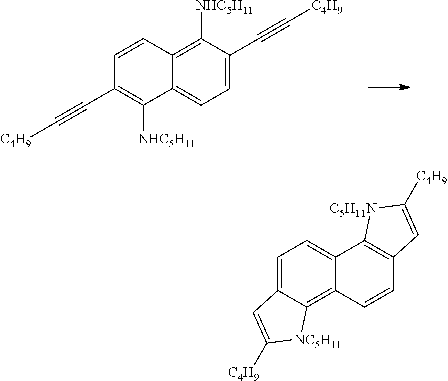

In an dried pressure tube, to a solution of 2,6-di(hex-1-yn-1-yl)-N₁,N₅-dipentylnaphthalene-1,5-diamine (580 mg) in DMSO (3 mL) was added finely crushed KOH (364 mg). The resulting reaction mixture was heated at 120° C. for 20 h and was extracted with ethyl acetate and the combined organic layer was dried (MgSO₄) and concentrated in vacuo. The residue was purified by column chromatography on silica gel to give 2,7-dibutyl-3,8-dipentyl-3,8-dihydroindolo[7,6-g]indole (70%). LRMS (ESI): Calcd. For C₃₂H₄₆N₂: 458.3661. Found: 458.4.

### Example 17

### 2,6-dibromonaphthalene-1,5-diol

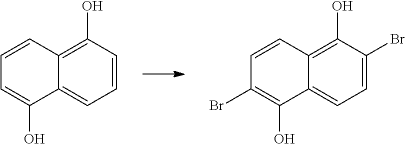 [SMILES: Oc1cccc2c(O)cccc12 ; Oc1c(Br)ccc2c(O)c(Br)ccc12]

To a suspension of naphthalene-1,5-diol (115.2 g) in CH₃CN (800 mL) was added DMF solution (400 mL) of NBS (254 g) dropwise and the mixture was stirred at room temperature and monitored by GCMS. Water was added quench the reaction. The resulting precipitate was collected by filtration and washed with water to give 2,6-dibromonaphthalene-1,5-diol (80%) LRMS (ESI): Calcd. For C₁₀H₆Br₂O₂: 317.9614. Found: 317.9.

### Example 18

### 2,6-dibromonaphthalene-1,5-diyl bis(trifluoromethanesulfonate)

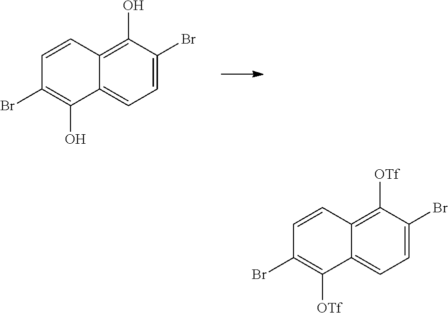 [SMILES: Oc1c(Br)ccc2c(O)c(Br)ccc12 ; O=S(=O)(Oc1c(Br)ccc2c(OS(=O)(=O)C(F)(F)F)c(Br)ccc12)C(F)(F)F]

To a suspension of 2,6-dibromonaphthalene-1,5-diol (4.3 g), pyridine (6.5 mL) in dichloromethane (130 mL) was slowly added trifluoromethanesulfonic anhydride (4.7 mL) at 0° C. After the mixture was stirred at room and monitored by GCMS. Water and hydrochloric acid (1 M) were added. The resulting mixture was extracted with dichloromethane and combined organic layer was dried (MgSO₄) and concentrated in vacuo. The residue was purified by column chromatography to give 2,6-dibromo-naphthalene-1,5-diyl bis(trifluoromethanesulfonate) (80%) LRMS (ESI): Calcd. For C₁₂H₄Br₂F₆O₆S₂: 582.0850. Found: 581.9.

### Example 19

### 2,6-dibromo-1,5-di(hex-1-yn-1-yl)naphthalene

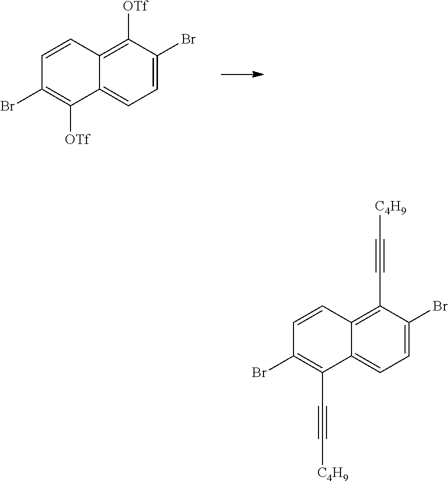

To a degassed solution of 2,6-dibromo-naphthalene-1,5-diyl bis(trifluoromethane-sulfonate) (3 g) in DMF (36 mL) and diisopropylamine (36 mL) was added Pd(PPh₃)₂Cl₂ (360 mg), CuI (196 mg) and hex-1-yne (1.25 mL). The reaction was stirred at room temperature and monitored by GCMS. Water and hydrochloric acid (1 M) were added. The resulting mixture was extracted with dichloromethane and the combined organic layer was dried (MgSO₄) and concentrated in vacuo. The residue was purified by column chromatography on silica gel eluted with hexane to give 2,6-dibromo-1,5-di(hex-1-yn-1-yl)naphthalene (71%) LRMS (ESI): Calcd. For C₂₂H₂₂Br₂: 446.2181. Found: 446.1.

### Example 20

### 1,5-di(hex-1-yn-1-yl)-N2,N6-dipentylnaphthalene-2,6-diamine

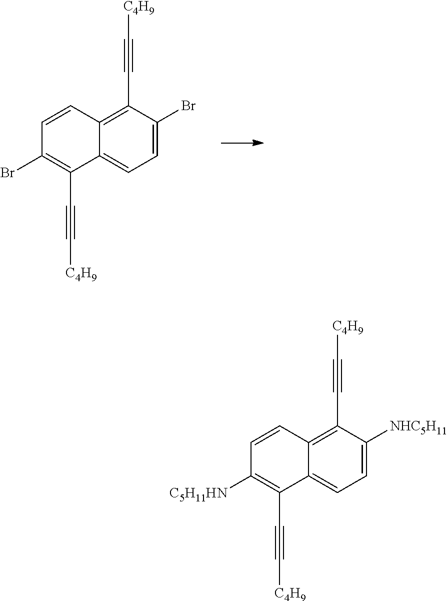

A solution of 2,6-dibromo-1,5-di(hex-1-yn-1-yl)naphthalene (1.42 g), t-BuONa (1.42 g), Pd₂dba₃ (146 mg) and (S)-BINAP (400 mg) in dry toluene (12 mL) was purged with nitrogen for 5 min. Pentan-1-amine (1.16 mL) was added via a syringe and the mixture was refluxed under nitrogen for 14 h. After cooling to room temperature, water was added to the solution and the reaction mixture was extracted twice with diethyl ether. After the organic phases were dried over MgSO4, the solvents were removed using a rotary evaporator. The crude product was purified by column chromatography and the desired product of 1,5-di(hex-1-yn-1-yl)-N₂,N₆-dipentylnaphthalene-2,6-diamine was obtained (70%). LRMS (ESI): Calcd. For C₃₂H₄₆N₂: 458.3661. Found: 458.4.

### Example 21

### 2,7-dibutyl-1,6-dipentyl-1,6-dihydroindolo[5,4-e]indole

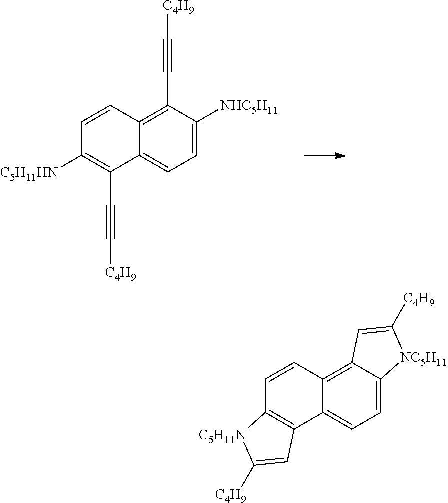

In an dried pressure tube, to a solution of 1,5-di(hex-1-yn-1-yl)-N₂,N₆-dipentylnaphthalene-2,6-diamine (100 mg) in DMSO (3 mL) was added finely crushed KOH (100 mg). The resulting reaction mixture was heated at 120° C. for 17 h and was extracted with ethyl acetate and the combined organic layer was dried (MgSO₄) and concentrated in vacuo. The residue was purified by column chromatography on silica gel to give 2,7-dibutyl-1,6-dipentyl-1,6-dihydroindolo[5,4-e]indole (50%). LRMS (ESI): Calcd. For C₃₂H₄₆N₂: 458.3661. Found: 458.3.

1. A compound of formula: 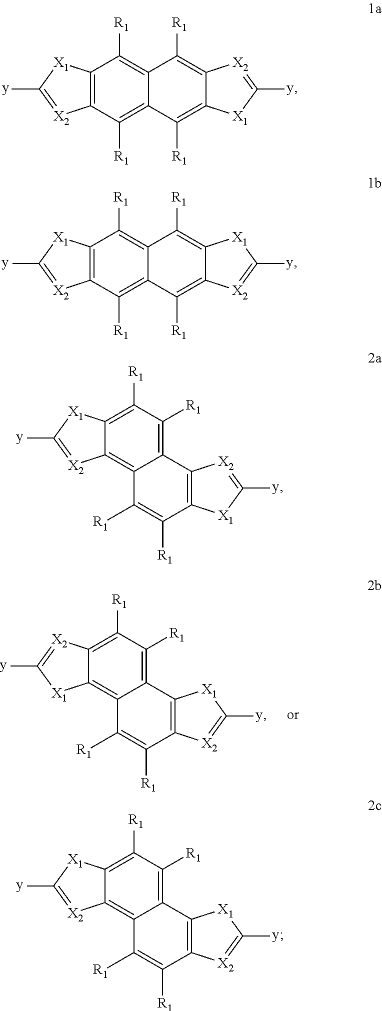
wherein each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; y is H, halo, trialkylsilane, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs; and
each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

2. The compound of claim 1, wherein the compound is 1a or 1b.

3. The compound of claim 1, wherein the compound is 2a, 2b, or 2c.

4. The compound of claim 1, wherein:
X₁ is S, Se, NR₁, PR₁, AsR₁, SbR₁, O, or Te, with the proviso that due to conjugation, X₁ may be bonded to one or more additional R₁;
X₂ is N or CR₁, with the proviso that due to conjugation, X₂ may be bonded to one or more additional R₁;
y is H, halo, trialkylsiane, optionally substituted C₁-C₄₀ alkyl, optionally substituted C₂-C₄₀ alkenyl, optionally substituted C₂-C₄₀ alkynyl, halo, OSO-alkyl, Mg-halo, Zn-halo, Sn(alkyl)₃, B(OH)₂, or B(alkoxy)₂; and
each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone.

5. The compound of claim 4, wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, optionally substituted cycloalkyl, optionally substituted cycloalkenyl, halo, optionally substituted heterocyclyl, or an optionally substituted aryl or optionally substituted heteroaryl from the group consisting of phenyl, thiophenyl, furanyl, pyrrolyl, imidazolyl, triazolyl, oxaxolyl, thiazolyl, pyridinyl, pyrimidinyl, triazinyl, naphthalenyl, isoquinolinyl, quinolinyl, or naphthyridinyl.

6. The compound of claim 1, wherein the hole reorganization energy is less than 0.35 eV.

7. The compound of claim 6, wherein the hole reorganization energy is from about 0.05 eV to about 0.35 eV.

8. (canceled)

9. (canceled)

10. (canceled)

11. (canceled)

12. (canceled)

13. (canceled)

14. A method of making a compound of structure: 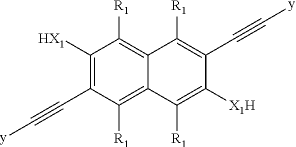
comprising allowing a compound of structure: 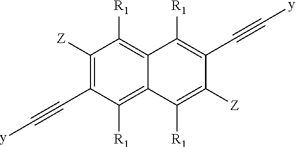
to undergo a substitution reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

15. A method of making a compound of structure: 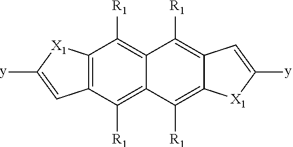
comprising allowing a compound of structure: 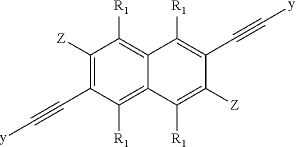
or structure: 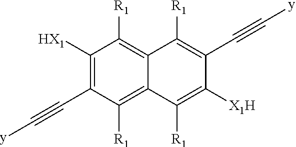
to undergo a ring cyclization reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each Z is independently Z₁ or I, each Z₁ is independently Cl or Br; and each y is independently H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

16. A method of making a compound of structure: 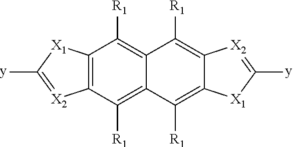
comprising allowing a compound of structure: 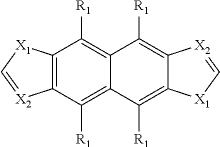
to undergo a substitution reaction,
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently CI or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

17. (canceled)

18. A method of making a compound of structure: 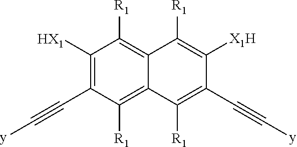
comprising allowing a compound of structure: 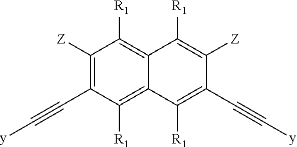
to undergo a substitution reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br.

19. A method of making a compound of structure: 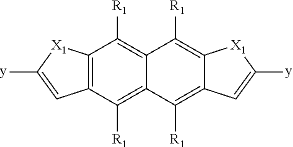
comprising allowing a compound of structure: 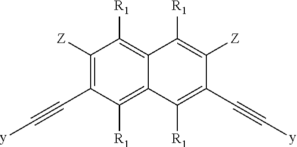
or structure: 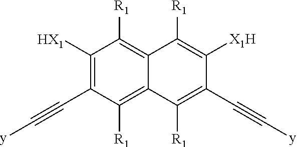
to undergo a ring cyclization reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

20. A method of making a compound of structure: 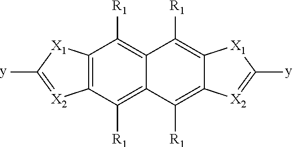
comprising allowing a compound of structure: 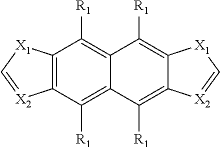
to undergo a substitution reaction,
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

21. (canceled)

22. A method of making a compound of structure: 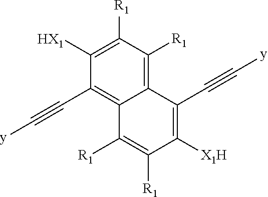
comprising allowing a compound of structure: 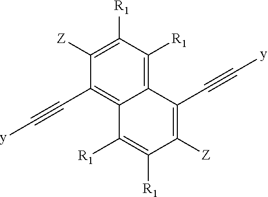
to undergo a substitution reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently CI or Brand each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

23. A method of making a compound of structure: 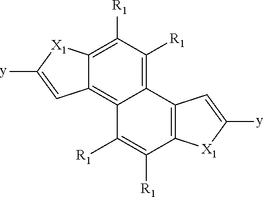
comprising allowing a compound of structure: 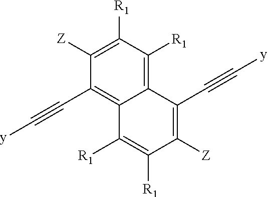
or structure: 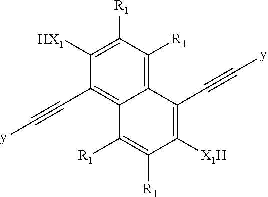
to undergo a ring cyclization reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

24. A method of making a compound of structure: 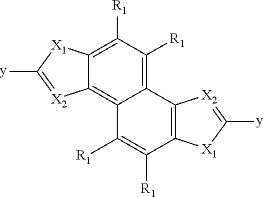
comprising allowing a compound of structure: 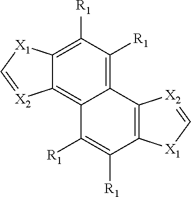
to undergo a substitution reaction,
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

25. (canceled)

26. A method of making a compound of structure: 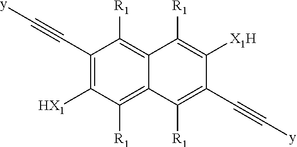
comprising allowing a compound of structure: 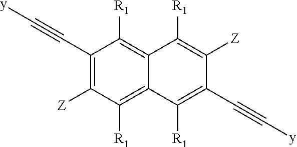
to undergo a substitution reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

27. A method of making a compound of structure: 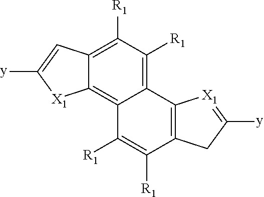
comprising allowing a compound of structure: 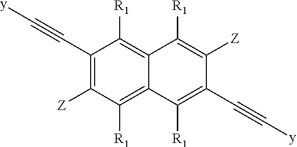
or structure: 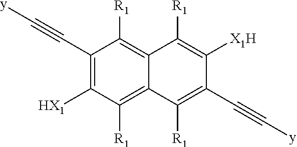
to undergo a ring cyclization reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide; optionally substituted sulfone, OSO-alkyl, Mg-halo, Zn-halo, Sn(alkyl)₃, B(OH)₂, B(alkoxy)₂, or OTs.

28. A method of making a compound of structure: 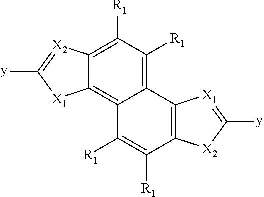
comprising allowing a compound of structure: 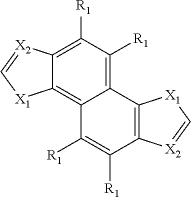
to undergo a substitution reaction,
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

29. (canceled)

30. A method of making a compound of structure: 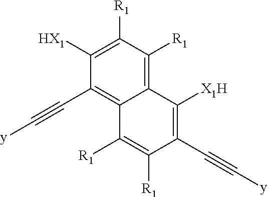
comprising allowing a compound of structure: 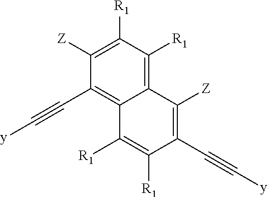
to undergo a substitution reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

31. A method of making a compound of structure: 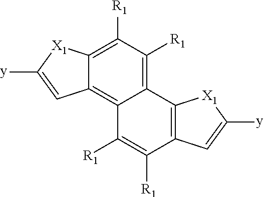
comprising allowing a compound of structure: 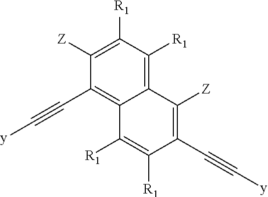
or structure: 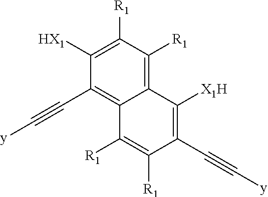
to undergo a ring cyclization reaction;
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; and each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

32. A method of making a compound of structure: 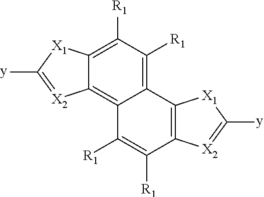
comprising allowing a compound of structure: 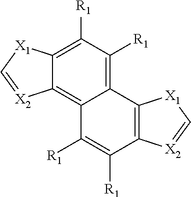
to undergo a substitution reaction,
wherein each R₁ is independently H, halo, optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, optionally substituted aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, or optionally substituted sulfone; each R₂ is independently optionally substituted C₁-C₄₀ alkyl or optionally substituted aryl; each X₁ is independently NR₁, PR₁, AsR₁, Sb, O, S, Te, or Se, with the proviso that due to conjugation, X₁ may be bonded to one or more additional R₁; each X₂ is independently N, P, As, SiR₁, or CR₁, with the proviso that due to conjugation, X₂ may be bonded to one or more additional R₁; each Z is independently Z₁ or I, each Z₁ is independently Cl or Br; and each y is independently is H, halo, trialkylsilane optionally substituted C₁-C₄₀ alkyl, optionally substituted aralkyl, alkoxy, alkylthio, optionally substituted C₂-C₄₀ alkenyl, optionally substituted C₂-C₄₀ alkynyl, aminocarbonyl, acylamino, acyloxy, aryl, aryloxy, optionally substituted amino, carboxyalkyl, optionally substituted cycloalkyl, optionally substituted cycloalkenyl, halo, acyl, optionally substituted heteroaryl, optionally substituted heteroaralkyl, heteroaryloxy, optionally substituted heterocyclyl, thiol, alkylthio, heteroarylthiol, optionally substituted sulfoxide, optionally substituted sulfone, OSO-alkyl, Mg-halo, Zn-halo, Sn(alkyl)₃, B(OH)₂, B(alkoxy)₂, or OTs.

33. (canceled)

34. A device comprising a compound of claim 1.

35. (canceled)
